(19) United States
(12) Patent Application Publication
(10) Pub. No.: US 2011/0263709 A1
(43) Pub. Date: Oct. 27, 2011

(54) METHOD FOR OBTAINING POLYUNSATURATED FATTY ACID-CONTAINING COMPOSITIONS FROM MICROBIAL BIOMASS

(75) Inventors: Keith W. Hutchenson, Lincoln University, PA (US); Richard E. Bockrath, Wilmington, DE (US); Robert D. Orlandi, Landenberg, PA (US)

(73) Assignee: E. I. DU PONT DE NEMOURS AND COMPANY, Wilmington, DE (US)

(21) Appl. No.: 13/090,373

(22) Filed: Apr. 20, 2011

Related U.S. Application Data
(60) Provisional application No. 61/326,793, filed on Apr. 22, 2010.

(51) Int. Cl. A61K 31/201; A23D 9/00; A61K 31/202

(52) U.S. Cl. 514/560; 426/601

(57) ABSTRACT

A method is disclosed for obtaining a refined lipid composition comprising at least one polyunsaturated fatty acid from a microbial biomass, wherein the refined lipid composition comprises at least one polyunsaturated fatty acid and is enriched in triacylglycerols relative to the oil composition of the microbial biomass.

This application claims the benefit of U.S. Provisional Application No. 61/326,793, filed Apr. 22, 2010, which is herein incorporated by reference in its entirety.

### FIELD OF THE INVENTION

The present invention relates to methods for obtaining a refined lipid composition, comprising at least one polyunsaturated fatty acid and enriched in triacylglyercols, by extraction of a disrupted microbial biomass with a solvent comprising carbon dioxide and fractionation.

### BACKGROUND OF THE INVENTION

There has been growing interest in including polyunsaturated fatty acids (PUFAs) such as eicosapentaenoic acid (EPA; omega-3) and docosahexaenoic acid (DHA; omega-3) in pharmaceutical and dietary products. PUFA-containing lipid compositions can be obtained, for example, from natural microbial sources, from recombinant microorganisms, or from fish oils and marine planktons. PUFA-containing lipid compositions are recognized as being oxidatively unstable under certain conditions, which necessitates expending considerable care to obtain un-oxidized compositions.

U.S. Pat. No. 4,675,132 discloses a process for the concentration of PUFA moieties in a fish oil containing relatively low proportions of saturated and monounsaturated fatty acid moieties of the same chain length as the PUFA moieties to be concentrated, which comprises transesterifying fish oil glycerides with a lower alkanol to form a mixture of lower alkyl fatty acid esters, and extracting said esters with carbon dioxide (CO₂) under supercritical conditions.

A process flow diagram developed for a continuous countercurrent supercritical CO₂ fractionation process that produces high concentration EPA is disclosed by V. J. Krukonis et al. (Adv. Seafood Biochem., Pap. Am. Chem. Soc. Annu. Meet. (1992), Meeting Date 1987, 169-179). The feedstock for the process is urea-crystallized ethyl esters of menhaden oil, and the basis for the design is a product concentration of 90% EPA (ethyl ester) at a yield of 90%.

U.S. Pat. No. 6,727,373 discloses a microbial PUFA-containing oil with a high triglyceride content and a high oxidative stability. In addition, a method is described for the recovery of such oil from a microbial biomass derived from a pasteurized fermentation broth, wherein the microbial biomass is subjected to extrusion to form granular particles, dried, and the oil is then extracted from the dried granules using an appropriate solvent.

Methods in which the distribution of triacylglycerols, diacylglycerols, monoacylglycerols, and free fatty acids can be adjusted in a PUFA-containing lipid composition are sought. Methods for obtaining PUFA-containing lipid compositions which have improved oxidative stability are desired. Methods for obtaining PUFA-containing lipid compositions enriched in triacylglycerols are also desired, as are economical methods for obtaining such compositions.

### SUMMARY OF THE INVENTION

In a first embodiment, the present invention is drawn to a method comprising the steps of:

a) processing an untreated disrupted microbial biomass having an oil composition comprising at least one polyunsaturated fatty acid with a solvent comprising liquid or supercritical fluid carbon dioxide to obtain:
(i) an extract comprising a lipid fraction substantially free of phospholipids; and,
(ii) a residual biomass comprising phospholipids; and,

b) fractionating the extract obtained in step (a), part (i) at least once to obtain a refined lipid composition comprising at least one polyunsaturated fatty acid, wherein the refined lipid composition is enriched in triacylglycerols relative to the oil composition of the untreated disrupted microbial biomass.

In a second embodiment, the refined lipid composition enriched in triacylglycerols comprises at least one lipid component selected from the group consisting of: diacylglycerols, monoacylglycerols, free fatty acids and combinations thereof.

In a third embodiment, the refined lipid composition enriched in triacylglycerols is enriched in at least one polyunsaturated fatty acid relative to the untreated disrupted microbial biomass.

In a fourth embodiment, the method of the invention further comprises a step selected from the group consisting of:
a) fractionating the extract obtained in step (a), part (i) to obtain a refined lipid composition comprising at least one polyunsaturated fatty acid, wherein the refined lipid composition is enriched in lipid components selected from the group consisting of diacylglycerols, monoacylglycerols, free fatty acids and combinations thereof relative to the oil composition of the untreated disrupted microbial biomass; and,
b) processing the residual biomass comprising phospholipids of step (a), part (ii) with an extractant to obtain a residual biomass extract consisting essentially of phospholipids.

In a fifth embodiment, the processing of step (a) is done at a temperature from about 20° C. to about 100° C. and at a pressure from about 60 bar to about 800 bar. The fractionating of step (b) is performed by altering the temperature, the pressure, or the temperature and the pressure, of the fractionating conditions.

In a sixth embodiment, the processing solvent of step (a) comprises supercritical fluid carbon dioxide and the fractionating of step (b) is done at a temperature from about 35° C. to about 100° C. and at a pressure from about 80 bar to about 600 bar.

In a seventh embodiment, the untreated disrupted microbial biomass comprises oleaginous microbial cells.

In an eighth embodiment, the at least one polyunsaturated fatty acid is selected from the group consisting of linoleic acid, γ-linolenic acid, eicosadienoic acid, dihomo-γ-linolenic acid, arachidonic acid, docosatetraenoic acid, ω-6 docosapentaenoic acid, α-linolenic acid, stearidonic acid, eicosatrienoic acid, eicosatetraenoic acid, ω-3 docosapentaenoic acid, docosahexaenoic acid, eicosapentaenoic acid, and mixtures thereof.

In a ninth embodiment, the residual biomass comprising phospholipids or the residual biomass extract consisting essentially of phospholipids is suitable for use as a component in an aquaculture feed

In a tenth embodiment, the untreated disrupted microbial biomass comprises at least 25 weight percent of eicosapentaenoic acid, measured as a weight percent of total fatty acids in the untreated disrupted microbial biomass.

In an eleventh embodiment, the present invention is drawn to a method comprising processing an untreated disrupted microbial biomass having an oil composition comprising at least one polyunsaturated fatty acid with a solvent comprising liquid or supercritical fluid carbon dioxide to obtain:
(i) an extract comprising a lipid fraction substantially free of phospholipids; and,
(ii) a residual biomass comprising phospholipids;

wherein said untreated disrupted microbial biomass is obtained from an oleaginous microorganism of the genus Yarrowia that accumulates in excess of 25% of its dry cell weight as oil; and,

wherein said oil composition comprising at least one polyunsaturated fatty acid comprises at least 25 weight percent of a polyunsaturated fatty acid having at least twenty carbon atoms and four or more carbon-carbon double bonds, measured as a weight percent of total fatty acids.

The untreated disrupted microbial biomass is preferably obtained from Yarrowia lipolytica and the at least one polyunsaturated fatty acid comprises eicosapentaenoic acid.

In a twelfth embodiment, the residual biomass comprising phospholipids is processed with an extractant to obtain a residual biomass extract consisting essentially of phospholipids.

### BIOLOGICAL DEPOSITS

The following biological materials have been deposited with the American Type Culture Collection (ATCC), 10801 University Boulevard, Manassas, Va. 20110-2209, and bear the following designations, accession numbers and dates of deposit.

| Biological Material | Accession No. | Date of Deposit |
|---|---|---|
| Yarrowia lipolytica Y4128 | ATCC PTA-8614 | Aug. 23, 2007 |
| Yarrowia lipolytica Y8412 | ATCC PTA-10026 | May 14, 2009 |
| Yarrowia lipolytica Y8259 | ATCC PTA-10027 | May 14, 2009 |

The biological materials listed above were deposited under the terms of the Budapest Treaty on the International Recognition of the Deposit of Microorganisms for the Purposes of Patent Procedure. The listed deposit will be maintained in the indicated international depository for at least 30 years and will be made available to the public upon the grant of a patent disclosing it. The availability of a deposit does not constitute a license to practice the subject invention in derogation of patent rights granted by government action.

Yarrowia lipolytica Y4305 was derived from Yarrowia lipolytica Y4128, according to the methodology described in U.S. Pat. Appl. Pub. No. 2009-0093543-A1. Yarrowia lipolytica Y9502 was derived from Yarrowia lipolytica Y8412, according to the methodology described in U.S. Pat. Appl. Pub. No. 2010-0317072-A1. Similarly, Yarrowia lipolytica Y8672 was derived from Yarrowia lipolytica Y8259, according to the methodology described in U.S. Pat. Appl. Pub. No. 2010-0317072-A1.

### BRIEF DESCRIPTION OF THE FIGURES

FIG. 1A and FIG. 1B provide an overview of the processes of the invention, in the form of a flowchart and should be viewed together when considering the description below. Each text box is assigned a letter label from A to L. Specifically, a microbial fermentation (FIG. 1A, I) results in untreated microbial biomass (FIG. 1A, B). Mechanical or chemical processing then produces an untreated disrupted microbial biomass (FIG. 1A, C). Oil extraction (FIG. 1A, D) of the untreated disrupted microbial biomass results in residual biomass comprising phospholipids (FIG. 1A, E) and an extracted oil substantially free of phospholipids (FIG. 1A, I), that may optionally be fractionated (FIG. 1B, I) to produce a refined lipid composition comprising at least one PUFA, wherein the refined lipid composition is enriched in TAGs (FIG. 1B, L) relative to the oil composition of the untreated disrupted microbial biomass.

FIG. 2 schematically illustrates one embodiment of the methods of the invention, in which microbial biomass is contacted with CO₂ to obtain an extract which is then fractionated.

FIG. 3 schematically illustrates one embodiment of the methods of the invention, in which microbial biomass is contacted with CO₂ to obtain an extract.

FIG. 4 is a graphical representation of the extraction curve obtained in Example 1.

### DETAILED DESCRIPTION OF THE INVENTION

The disclosures of all patent and non-patent literature cited herein are hereby incorporated by reference in their entireties.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises”, “comprising”, “includes”, “including”, “has”, “having”, “contains”, “containing” or any other variation thereof, are intended to cover a non-exclusive inclusion. For example, a composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e., occurrences) of the element or component. Therefore “a” or “an” should be read to include one or at least one, and the singular word form of the element or component also includes the plural unless the number is obviously meant to be singular.

As used herein the term “invention” or “present invention” is intended to refer to all aspects and embodiments of the invention as described in the claims and specification herein and should not be read so as to be limited to any particular embodiment or aspect.

The following definitions are used in this disclosure:

“Supercritical fluid” is abbreviated as “SCF”.

“Carbon dioxide” is abbreviated as “CO₂”.

“American Type Culture Collection” is abbreviated as “ATCC”.

“Polyunsaturated fatty acid(s)” is abbreviated as “PUFA(s)”.

“Phospholipids” are abbreviated as “PLs”.

“Monoacylglycerols” are abbreviated as “MAGs”.

“Diacylglycerols” are abbreviated as “DAGs”.

“Triacylglycerols” are abbreviated as “TAGs”. Herein the term “triacylglycerols” (TAGs) is synonymous with the term “triacylglycerides” and refers to neutral lipids composed of three fatty acyl residues esterified to a glycerol molecule. TAGs can contain long chain PUFAs and saturated fatty acids, as well as shorter chain saturated and unsaturated fatty acids.

“Free fatty acids” are abbreviated as “FFAs”.

“Total fatty acids” are abbreviated as “TFAs”.

“Fatty acid methyl esters” are abbreviated as “FAMEs”.

“Dry cell weight” is abbreviated as “DCW”.

“Weight percent” is abbreviated as “wt %”.

As used herein the term “microbial biomass” refers to microbial cellular material from a microbial fermentation of oil-containing microbes that is conducted to produce microbial oil comprising PUFAs. The microbial biomass may be in the form of whole cells, whole cell lysates, homogenized cells, partially hydrolyzed cellular material, and/or disrupted cells (thus the term microbial biomass may generically refer to untreated microbial biomass or untreated disrupted microbial biomass, infra).

The term “untreated microbial biomass” refers to microbial biomass prior to extraction with a solvent. The microbial biomass may optionally be e.g., de-watered, dried, pelletized and/or granulated. The terms “untreated microbial biomass” and “unrefined microbial biomass” are used interchangeably herein.

The term “untreated disrupted microbial biomass” refers to microbial biomass that has been subjected to a process of disruption and that has not been subjected to extraction with a solvent. As one of skill in the art will appreciate, numerous processes of cell disruption are available, including, for example, chemical processes for cellular lysing or mechanical disruption via physical means such as bead beaters, screw extrusion, etc.

The term “residual biomass” refers to microbial cellular material obtained from the fermentation of oil-containing microbes that has been subjected to extraction at least once with a solvent. Thus, the residual biomass is spent microbial biomass from which PUFA-containing microbial oil has been removed by extraction.

The term “enriched” means having a larger quantity, for example a quantity only slightly more than the original quantity, or for example a quantity exponentially greater than the original quantity, and including all quantities in between.

The term “reduced” or “depleted” means having a smaller quantity, for example a quantity only slightly less than the original quantity, or for example a quantity completely lacking in the specified material, and including all quantities in between.

The term “lipids” refer to any fat-soluble (i.e., lipophilic), naturally-occurring molecule. Lipids are a diverse group of compounds that have many key biological functions, such as structural components of cell membranes, energy storage sources and intermediates in signaling pathways. Lipids may be broadly defined as hydrophobic or amphiphilic small molecules that originate entirely or in part from either ketoacyl or isoprene groups. A general overview of lipids, based on the Lipid Metabolites and Pathways Strategy (LIPID MAPS) classification system (National Institute of General Medical Sciences, Bethesda, Md.), is shown below in Table 1.

TABLE 1
Overview Of Lipid Classes

| Structural | | |
|---|---|---|
| Building Block | Lipid Category | Examples Of Lipid Classes |
| Derived from | Fatty Acyls | Includes fatty acids, eicosanoids, fatty |
| condensation | | esters and fatty amides |
| of ketoacyl | Glycerolipids | Includes mainly mono-, di- and tri- |
| subunits | | substituted glycerols, the most well-known |
| | | being the fatty acid esters of glycerol |
| | | (triacylglycerols) |
| | Glycero- | Includes phosphatidylcholine, |
| | phospholipids or | phosphatidylethanolamine, phospha- |
| | Phospholipids | tidylserine, phosphatidylinositols and |
| | | phosphatidic acids |
| | Sphingolipids | Includes ceramides, phospho-sphingolipids |
| | | (e.g., sphingomyelins), glycosphingolipids |
| | | (e.g., gangliosides), sphingosine, |
| | | cerebrosides |
| | Saccharolipids | Includes acylaminosugars, acylamino-sugar |
| | | glycans, acyltrehaloses, |
| | | acyltrehalose glycans |
| | Polyketides | Includes halogenated acetogenins, |
| | | polyenes, linear tetracyclines, |
| | | polyether antibiotics, flavonoids, |
| | | aromatic polyketides |
| Derived from | Sterol Lipids | Includes sterols (e.g., cholesterol), C18 |
| condensation | | steroids (e.g., estrogens), C19 steroids |
| of isoprene | | (e.g., androgens), C21 steroids (e.g., |
| subunits | | progestogens, glucocorticoids and mineral- |
| | | ocorticoids), secosteroids, bile acids |
| | Prenol Lipids | Includes isoprenoids, carotenoids, quinones, |
| | | hydroquinones, polyprenols, hopanoids |

The term “oil” refers to a lipid substance that is liquid at 25° C. and usually polyunsaturated. In oleaginous organisms, oil constitutes a major part of the total lipid. “Oil” is composed primarily of triacylglycerols (TAGs) but may also contain other neutral lipids, phospholipids (PLs) and free fatty acids (FFAs). The fatty acid composition in the oil and the fatty acid composition of the total lipid are generally similar; thus, an increase or decrease in the concentration of PUFAs in the total lipid will correspond with an increase or decrease in the concentration of PUFAs in the oil, and vice versa.

“Neutral lipids” refer to those lipids commonly found in cells in lipid bodies as storage fats and are so called because at cellular pH, the lipids bear no charged groups. Generally, they are completely non-polar with no affinity for water. Neutral lipids generally refer to mono-, di-, and/or triesters of glycerol with fatty acids, also called monoacylglycerols (MAGs), diacylglycerols (DAGs) or TAGs, respectively, or collectively, acylglycerols. A hydrolysis reaction must occur to release FFAs from acylglycerols.

The term “extraction” refers to a physical or chemical method of removing one or more components from a substrate by means of a solvent.

The term “fractionation” refers to the selective separation of the components of a complex mixture of molecules into fractions having distributions of these components that are different from that of the starting material and from each other.

The term “extracted oil” refers to an oil that has been separated from cellular materials, such as the microorganism in which the microbial oil was synthesized. Extracted oils are obtained through a wide variety of methods, the simplest of which involves physical means alone. For example, mechanical crushing using various press configurations (e.g., screw, expeller, piston, bead beaters, etc.) can separate oil from cellular materials. Alternatively, oil extraction can occur via treatment with various organic solvents (e.g., hexane, iso-hexane), via enzymatic extraction, via osmotic shock, via ultrasonic extraction, via supercritical fluid extraction (e.g., CO₂ extraction), via saponification and via combinations of these methods. Further purification or concentration of an extracted oil is optional.

The term “refined lipid composition” refers to a microbial oil composition that is the product of the extraction and fractionation methods disclosed herein. Thus, the refined lipid composition is an extracted oil substantially free of phospholipids. Although one of skill in the art will appreciate that various fractions can be separated in a fractionation process, at least one refined lipid composition resulting from the fractionation will be enriched in TAGs relative to the oil composition of the microbial biomass. The refined lipid composition enriched in TAGs may comprise DAGs, MAGs, FFAs and combinations thereof. Additional refined lipid compositions may be separated comprising various fractions of neutral lipids, FFAs and combinations thereof, such as a refined lipid composition enriched in lipid components selected from the group consisting of DAGs, MAGs, FFAs and combinations thereof. The refined lipid composition(s) may undergo further purification to produce “purified oil”.

The term “substantially free of phospholipids (PLs)” means comprising no more than about 0.1 weight percent of phospholipids. Thus, an extract comprising a lipid fraction is substantially free of PLs when the concentration of PLs is no more than about 0.1 wt %, measured as a wt % of the total lipids. Similarly, a refined lipid composition is substantially free of PLs when the concentration of PLs is no more than about 0.1 wt %, measured as a wt % of the total lipids.

The term “total fatty acids” (TFAs) herein refer to the sum of all cellular fatty acids that can be derivatized to fatty acid methyl esters (FAMEs) by the base transesterification method (as known in the art) in a given sample, which may be the microbial biomass or oil, for example. Thus, total fatty acids include fatty acids from neutral lipid fractions (including DAGs, MAGs and TAGs) and from polar lipid fractions (including the phosphatidylcholine and the phosphatidylethanolamine fractions) but not FFAs.

The term “total lipid content” of cells is a measure of TFAs as a percent of the dry cell weight (DCW), although total lipid content can be approximated as a measure of FAMEs as a percent of the DCW (FAMEs % DCW). Thus, total lipid content (TFAs % DCW) is equivalent to, e.g., milligrams of total fatty acids per 100 milligrams of DCW.

The concentration of a fatty acid in the total lipid is expressed herein as a weight percent of TFAs (% TFAs), e.g., milligrams of the given fatty acid per 100 milligrams of TFAs. Unless otherwise specifically stated in the disclosure herein, reference to the percent of a given fatty acid with respect to total lipids is equivalent to concentration of the fatty acid as % TFAs (e.g., % EPA of total lipids is equivalent to EPA % TFAs).

In some cases, it is useful to express the content of a given fatty acid(s) in a cell as its weight percent of the dry cell weight (% DCW). Thus, for example, EPA % DCW would be determined according to the following formula: (EPA % TFAs)*(TFAs % DCW)]/100. The content of a given fatty acid(s) in a cell as its weight percent of the dry cell weight (% DCW) can be approximated, however, as: (EPA % TFAs)*(FAMEs % DCW)]/100.

The terms “lipid profile” and “lipid composition” are interchangeable and refer to the amount of individual fatty acids contained in a particular lipid fraction, such as in the total lipid or the oil, wherein the amount is expressed as a weight percent of TFAs. The sum of each individual fatty acid present in the mixture should be 100.

The term “fatty acids” refers to long chain aliphatic acids (alkanoic acids) of varying chain lengths, from about C₁₂ to C₂₂, although both longer and shorter chain-length acids are known. The predominant chain lengths are between C₁₆ and C₂₂. The structure of a fatty acid is represented by a simple notation system of “X:Y”, where X is the total number of carbon [“C”] atoms in the particular fatty acid and Y is the number of double bonds. Additional details concerning the differentiation between “saturated fatty acids” versus “unsaturated fatty acids”, “monounsaturated fatty acids” versus “polyunsaturated fatty acids” (PUFAs), and “omega-6 fatty acids” (“ω-6” or “n-6”) versus “omega-3 fatty acids” (“ω-3” or “n-3”) are provided in U.S. Pat. No. 7,238,482, which is hereby incorporated herein by reference.

Nomenclature used to describe PUFAs herein is given in Table 2. In the column titled “Shorthand Notation”, the omega-reference system is used to indicate the number of carbons, the number of double bonds and the position of the double bond closest to the omega carbon, counting from the omega carbon, which is numbered 1 for this purpose. The remainder of the Table summarizes the common names of omega-3 and omega-6 fatty acids and their precursors, the abbreviations that will be used throughout the specification and the chemical name of each compound.

TABLE 2
Nomenclature of Polyunsaturated Fatty Acids And Precursors

| | | | Shorthand |
|---|---|---|---|
| Common Name | Abbreviation | Chemical Name | Notation |
| Myristic | — | tetradecanoic | 14:0 |
| Palmitic | Palmitate | hexadecanoic | 16:0 |
| Palmitoleic | — | 9-hexadecenoic | 16:1 |
| Stearic | — | octadecanoic | 18:0 |
| Oleic | — | cis-9-octadecenoic | 18:1 |
| Linoleic | LA | cis-9,12-octadecadienoic | 18:2 ω-6 |
| γ-Linolenic | GLA | cis-6,9,12-octadecatrienoic | 18:3 ω-6 |
| Eicosadienoic | EDA | cis-11,14-eicosadienoic | 20:2 ω-6 |
| Dihomo-γ- | DGLA or | cis-8,11,14-eicosatrienoic | 20:3 ω-6 |
| Linolenic | HGLA | | |
| Arachidonic | ARA | cis-5,8,11,14- | 20:4 ω-6 |
| | | eicosatetraenoic | |
| α-Linolenic | ALA | cis-9,12,15- | 18:3 ω-3 |
| | | octadecatrienoic | |
| Stearidonic | STA | cis-6,9,12,15- | 18:4 ω-3 |
| | | octadecatetraenoic | |
| Eicosatrienoic | ETrA | cis-11,14,17-eicosatrienoic | 20:3 ω-3 |
| Eicosa- | ETA | cis-8,11,14,17- | 20:4 ω-3 |
| tetraenoic | | eicosatetraenoic | |
| Eicosa- | EPA | cis-5,8,11,14,17- | 20:5 ω-3 |
| pentaenoic | | eicosapentaenoic | |
| Docosa- | DTA | cis-7,10,13,16- | 22:4 ω-3 |
| tetraenoic | | docosatetraenoic | |
| Docosa- | DPAn-6 | cis-4,7,10,13,16- | 22:5 ω-6 |
| pentaenoic | | docosapentaenoic | |
| Docosa- | DPAn-3 | cis-7,10,13,16,19- | 22:5 ω-3 |
| pentaenoic | | docosapentaenoic | |
| Docosa- | DHA | cis-4,7,10,13,16,19- | 22:6 ω-3 |
| hexaenoic | | docosahexaenoic | |

The term “high-level PUFA production” refers to production of at least about 25% PUFA in the total lipids of the microbial host, preferably at least about 30% PUFA in the total lipids, more preferably at least about 35% PUFA in the total lipids, more preferably at least about 40% PUFA in the total lipids, more preferably at least about 40-45% PUFA in the total lipids, more preferably at least about 45-50% PUFA in the total lipids, more preferably at least about 50-60%, and most preferably at least about 60-70% PUFA in the total lipids. The structural form of the PUFA is not limiting; thus, for example, the PUFAs may exist in the total lipids as FFAs or in esterified forms such as acylglycerols, phospholipids, sulfolipids or glycolipids.

The term “oil-containing microbe” refers to a microorganism capable of producing microbial oil. Thus, an oil-containing microbe may be yeast, algae, euglenoids, stramenopiles, fungi, or combinations thereof. In preferred embodiments, the oil-containing microbe is oleaginous.

The term “oleaginous” refers to those organisms that tend to store their energy source in the form of oil (Weete, In: Fungal Lipid Biochemistry, 2^{nd} Ed., Plenum, 1980). Generally, the cellular oil of oleaginous microorganisms follows a sigmoid curve, wherein the concentration of lipid increases until it reaches a maximum at the late logarithmic or early stationary growth phase and then gradually decreases during the late stationary and death phases (Yongmanitchai and Ward, Appl. Environ. Microbiol., 57:419-25 (1991)). It is not uncommon for oleaginous microorganisms to accumulate in excess of about 25% of their dry cell weight as oil. Examples of oleaginous organisms include, but are not limited to organisms from a genus selected from the group consisting of Mortierella, Thraustochytrium, Schizochytrium and various oleaginous yeast.

The term “oleaginous yeast” refers to those microorganisms classified as yeasts that can make oil. Examples of oleaginous yeast include, but are by no means limited to, the following genera: Yarrowia, Candida, Rhodotorula, Rhodosporidium, Cryptococcus, Trichosporon and Lipomyces.

The term “animal feed” refers to feeds intended exclusively for consumption by animals, including domestic animals such as pets, farm animals, etc. or for animals raised for the production of food, such as for e.g., fish farming. The terms “aquaculture feed”, “aquafeed” and “feed nutrient” are as defined in U.S. Pat. Appl. Pub. No. 2006-0115881-A1.

In general, lipid accumulation in oleaginous microorganisms is triggered in response to the overall carbon to nitrogen ratio present in the growth medium. This process, leading to the de novo synthesis of free palmitate (16:0) in oleaginous microorganisms, is described in detail in U.S. Pat. No. 7,238,482. Palmitate is the precursor of longer-chain saturated and unsaturated fatty acid derivates, which are formed through the action of elongases and desaturases.

A wide spectrum of fatty acids (including saturated and unsaturated fatty acids and short-chain and long-chain fatty acids) can be incorporated into TAGs, the primary storage unit for fatty acids. In the methods described herein, incorporation of long chain PUFAs into TAGs is most desirable, although the structural form of the PUFA is not limiting (thus, for example, EPA may exist in the total lipids as FFAs or in esterified forms such as acylglycerols, PLs, sulfolipids or glycolipids). More specifically, in one embodiment of the present methods, the oil-containing microbes will produce at least one PUFA selected from the group consisting of LA, GLA, EDA, DGLA, ARA, DTA, DPAn-6, ALA, STA, ETrA, ETA, EPA, DPAn-3, DHA and mixtures thereof. More preferably, the at least one PUFA has at least a C₂₀ chain length, such as PUFAs selected from the group consisting of EDA, DGLA, ARA, DTA, DPAn-6, ETrA, ETA, EPA, DPAn-3, DHA, and mixtures thereof. In another embodiment, the at least one PUFA has at least a C₂₀ chain length and four or more carbon-carbon double bonds, i.e., a PUFA selected from the group consisting of ARA, EPA, DPAn-6, DPAn-3, DHA and mixtures thereof. In another preferred embodiment, the at least one PUFA is selected from the group consisting of EPA and DHA.

Although most PUFAs are incorporated into TAGs as neutral lipids and are stored in lipid bodies, it is important to note that a measurement of the total PUFAs within an oleaginous organism should minimally include those PUFAs that are located in the phosphatidylcholine, phosphatidylethanolamine and TAG fractions.

In one embodiment herein, the present invention relates to a method for obtaining a refined lipid composition comprising at least one PUFA, wherein the refined lipid composition is enriched in TAGs relative to the oil composition of the untreated disrupted microbial biomass. The refined lipid composition enriched in TAGs may further comprise DAGs, MAGs, FFAs and combinations thereof. Additional refined lipid composition fraction(s) may be obtained, comprising at least one PUFA and enriched in DAGs, MAGs, FFAs, and combinations thereof. Preferably, the at least one refined lipid composition enriched in TAGs is depleted in free FFAs relative to the oil composition of the microbial biomass and enriched in at least one PUFA relative to the untreated disrupted microbial biomass. Most preferably, the enriched at least one PUFA has at least 20 or more carbon atoms.

In an alternate embodiment herein, the present invention relates to a method wherein untreated disrupted microbial biomass is treated with a solvent comprising liquid or supercritical fluid carbon dioxide, wherein: (a) the untreated disrupted microbial biomass is obtained from an oleaginous microorganism of the genus Yarrowia that accumulates in excess of 25% of its dry cell weight as oil; and, (b) the oil composition comprising at least one PUFA comprises at least 25 weight percent of a PUFA having at least twenty carbon atoms and four or more carbon-carbon double bonds, measured as a weight percent of total fatty acids. This results in: (i) an extract comprising a lipid fraction substantially free of phospholipids; and, (ii) a residual biomass comprising phospholipids.

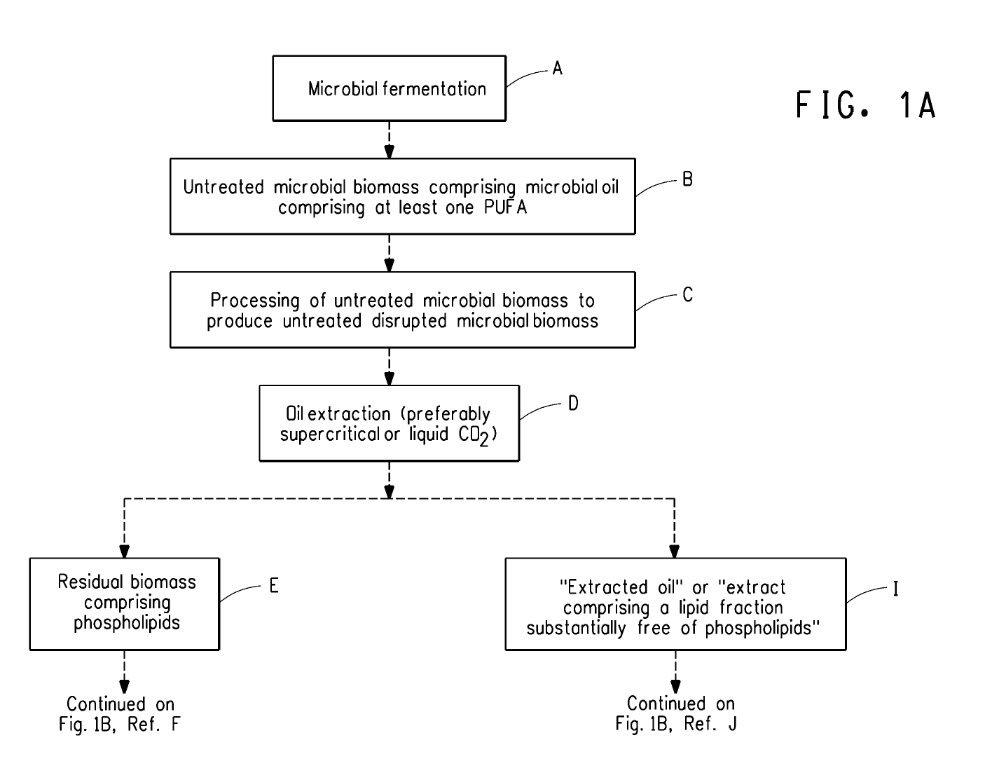

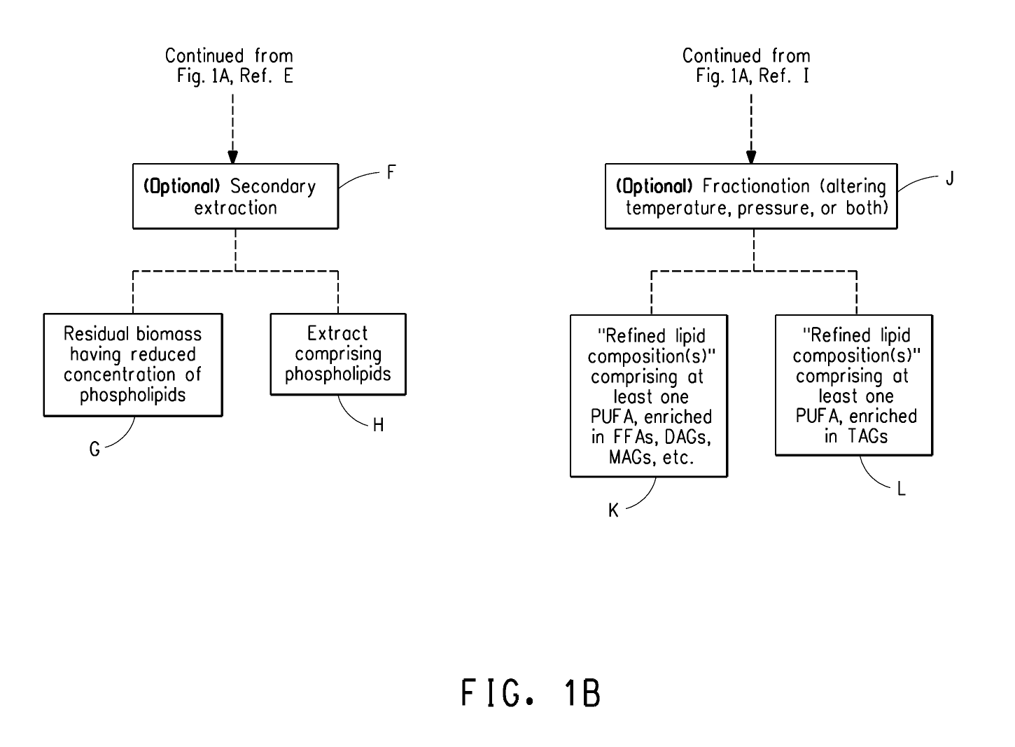

Although the present invention is broadly drawn to methods as disclosed herein, one will appreciate an overview of the related processes that may be useful to obtain the oil-containing microbes themselves from which the untreated disrupted microbial biomass is obtained (see FIG. 1A and FIG. 1B, and references to text boxes therein). Most processes will begin with a microbial fermentation (FIG. 1A, A), wherein a particular microorganism is cultured under conditions that permit growth and production of microbial oils comprising at least one PUFA. At an appropriate time, the microbial cells are harvested from the fermentation vessel. This untreated microbial biomass (FIG. 1A, B) may optionally be processed using various means, such as dewatering, drying, pelletization, granulation, etc., prior to undergoing a process of disruption (FIG. 1A, C). Oil extraction (FIG. 1A, D) of the untreated disrupted microbial biomass is then performed, producing residual biomass comprising phospholipids [“PLs”] (e.g., cell debris) (FIG. 1A, E) and an extracted oil substantially free of PLs (FIG. 1A, I), that may optionally be fractionated (FIG. 1B, J) to produce a refined lipid composition comprising at least one PUFA, wherein the refined lipid composition is enriched in TAGs (FIG. 1B, L) relative to the oil composition of the untreated disrupted microbial biomass. The residual biomass comprising PLs (FIG. 1A, E) may be further extracted (FIG. 1B, F). Each of these aspects of FIG. 1A and FIG. 1B will be discussed in further detail below.

Oil-containing microbes produce microbial biomass via microbial fermentation. The microbial biomass may be from any microorganism, whether naturally occurring or recombinant (“genetically engineered”), capable of producing a microbial oil comprising at least one PUFA. Thus, for example, oil-containing microbes may be selected from the group consisting of yeast, algae, euglenoids, stramenopiles, fungi, and mixtures thereof. Preferably, the microorganism will be capable of high level PUFA production within the microbial oil.

As an example, commercial sources of ARA oil are typically produced from microorganisms in the genera Mortierella (filamentous fungus), Entomophthora, Pythium and Porphyridium (red alga). Most notably, Martek Biosciences Corporation (Columbia, Md.) produces an ARA-containing fungal oil (ARASCO®; U.S. Pat. No. 5,658,767) which is substantially free of EPA and which is derived from either Mortierella alpina or Pythium insidiuosum.

Similarly, EPA can be produced microbially via numerous different processes based on the natural abilities of the specific microbial organism utilized [e.g., heterotrophic diatoms Cyclotella sp. and Nitzschia sp. (U.S. Pat. No. 5,244,921); Pseudomonas, Alteromonas or Shewanella species (U.S. Pat. No. 5,246,841); filamentous fungi of the genus Pythium (U.S. Pat. No. 5,246,842); or Mortierella elongata, M. exigua, or M. hygrophila (U.S. Pat. No. 5,401,646)]. A useful review describing microorganisms naturally producing EPA is that of Z. Wen and F. Chen, In Single Cell Oils; C. Ratledge and Z. Cohen, Eds.; AOCS Publishing, 2005; Chapter 10, entitled “Prospects for EPA production using microorganisms”.

DHA can also be produced using processes based on the natural abilities of the native microbe. See, e.g., processes developed for Schizochytrium species (U.S. Pat. No. 5,340,742; U.S. Pat. No. 6,582,941); Ulkenia (U.S. Pat. No. 6,509,178); Pseudomonas sp. YS-180 (U.S. Pat. No. 6,207,441); Thraustochytrium genus strain LFF1 (U.S. Pat. Appl. Pub. No. 2004/0161831 A1); Crypthecodinium cohnii (U.S. Pat. Appl. Pub. No. 2004/0072330 A1; de Swaaf, M. E. et al. Biotechnol Bioeng., 81 (6):666-72 (2003) and Appl Microbiol Biotechnol., 61 (1):40-3 (2003)); Emiliania sp. (Japanese Patent Publication (Kokai) No. 5-308978 (1993)); and Japonochytrium sp. (ATCC #28207; Japanese Patent Publication (Kokai) No. 199588/1989)]. Additionally, the following microorganisms are known to have the ability to produce DHA: Vibrio marinus (a bacterium isolated from the deep sea; ATCC #15381); the micro-algae Cyclotella cryptica and Isochrysis galbana; and, flagellate fungi such as Thraustochytrium aureum (ATCC #34304; Kendrick, Lipids, 27:15 (1992)) and the Thraustochytrium sp. designated as ATCC #28211, ATCC #20890 and ATCC #20891. Currently, there are at least three different fermentation processes for commercial production of DHA: fermentation of C. cohnii for production of DHASCO™ (Martek Biosciences Corporation, Columbia, Md.); fermentation of Schizochytrium sp. for production of an oil formerly known as DHAGold (Martek Biosciences Corporation); and fermentation of Ulkenia sp. for production of DHActive™ (Nutrinova, Frankfurt, Germany).

Microbial production of PUFAs in microbial oils using recombinant means is expected to have several advantages over production from natural microbial sources. For example, recombinant microbes having preferred characteristics for oil production can be used, since the naturally occurring microbial fatty acid profile of the host can be altered by the introduction of new biosynthetic pathways in the host and/or by the suppression of undesired pathways, thereby resulting in increased levels of production of desired PUFAs (or conjugated forms thereof) and decreased production of undesired PUFAs. Secondly, recombinant microbes can provide PUFAs in particular forms which may have specific uses. Additionally, microbial oil production can be manipulated by controlling culture conditions, notably by providing particular substrate sources for microbially expressed enzymes, or by addition of compounds/genetic engineering to suppress undesired biochemical pathways. Thus, for example, it is possible to modify the ratio of omega-3 to omega-6 fatty acids so produced, or engineer production of a specific PUFA (e.g., EPA) without significant accumulation of other PUFA downstream or upstream products.

Thus, for example, a microbe lacking the natural ability to make EPA can be engineered to express a PUFA biosynthetic pathway by introduction of appropriate PUFA biosynthetic pathway genes, such as specific combinations of delta-5 desaturases, delta-6 desaturases, delta-12 desaturases, delta-15 desaturases, delta-17 desaturases, delta-9 desaturases, delta-8 desaturases, delta-9 elongases, C_{14/16} elongases, C_{16/18} elongases and C_{18/20} elongases, although it is to be recognized that the specific enzymes (and genes encoding those enzymes) introduced are by no means limiting to the invention herein.

As an example, several types of yeast have been recombinantly engineered to produce at least one PUFA. See for example, work in Saccharomyces cerevisiae (Dyer, J. M. et al., Appl. Environ. Microbiol., 59:224-230 (2002); Domergue, F. et al., Eur. J. Biochem., 269:4105-4113 (2002); U.S. Pat. No. 6,136,574; U.S. Pat. Appl. Pub. No. 2006-0051847-A1) and the oleaginous yeast, Yarrowia lipolytica (U.S. Pat. No. 7,238,482; U.S. Pat. No. 7,465,564; U.S. Pat. No. 7,588,931; U.S. Pat. Appl. Pub. No. 2006-0115881-A1; U.S. Pat. No. 7,550,286; U.S. Pat. Appl. Pub. No. 2009-0093543-A1; U.S. Pat. Appl. Pub. No. 2010-0317072-A1).

In some embodiments, advantages are perceived if the microbial host cells are oleaginous. The oleaginous microbial host cells may be e.g., a member of a genus selected from the group consisting of Mortierella, Thraustochytrium, Schizochytrium and oleaginous yeast. Oleaginous yeast are naturally capable of oil synthesis and accumulation, wherein the total oil content can comprise greater than about 25% of the cellular dry weight, more preferably greater than about 30% of the cellular dry weight, and most preferably greater than about 40% of the cellular dry weight. In alternate embodiments, a non-oleaginous yeast can be genetically modified to become oleaginous such that it can produce more than 25% oil of the cellular dry weight, e.g., yeast such as Saccharomyces cerevisiae (Int'l. App. Pub. No. WO 2006/102342).

Genera typically identified as oleaginous yeast include, but are not limited to: Yarrowia, Candida, Rhodotorula, Rhodosporidium, Cryptococcus, Trichosporon and Lipomyces. More specifically, illustrative oil-synthesizing yeasts include: Rhodosporidium toruloides, Lipomyces starkeyii, L. lipoferus, Candida revkaufi, C. pulcherrima, C. tropicalis, C. utilis, Trichosporon pullans, T. cutaneum, Rhodotorula glutinus, R. graminis, and Yarrowia lipolytica (formerly classified as Candida lipolytica).

Most preferred is the oleaginous yeast Yarrowia lipolytica; and, in a further embodiment, most preferred are the Y. lipolytica strains designated as ATCC #20362, ATCC #8862, ATCC #18944, ATCC #76982 and/or LGAM S(7)1 (Papanikolaou S., and Aggelis G., Bioresour. Technol. 82 (1):43-9 (2002)).

In some embodiments, it may be desirable for the oleaginous yeast to be capable of “high-level PUFA production”, wherein the organism can produce at least about 5-10% of the desired PUFA (i.e., LA, ALA, EDA, GLA, STA, ETrA, DGLA, ETA, ARA, DPA n-6, EPA, DPA n-3 and/or DHA) in the total lipids. More preferably, the oleaginous yeast will produce at least about 10-25% of the desired PUFA in the total lipids, more preferably at least about 25-35% of the desired PUFA in the total lipids, more preferably at least about 35-50% of the desired PUFA in the total lipids, and most preferably at least about 50-70% of the desired PUFA in the total lipids. The structural form of the PUFA is not limiting; thus, for example, EPA may exist in the total lipids as FFAs or in esterified forms. Preferably, the at least one PUFA is in the form of TAGs.

Thus, the PUFA biosynthetic pathway genes and gene products described herein may be produced in heterologous microbial host cells, particularly in the cells of oleaginous yeasts (e.g., of the genus Yarrowia). Expression in recombinant microbial hosts may be useful for the production of various PUFA pathway intermediates, or for the modulation of PUFA pathways already existing in the host for the synthesis of new products heretofore not possible using the host.

Although numerous oleaginous yeast could be engineered for production of a preferred omega-3/omega-6 PUFA(s) based on the cited teachings provided above, representative PUFA-producing strains of the oleaginous yeast Yarrowia lipolytica are described in Table 3. These strains possess various combinations of the following PUFA biosynthetic pathway genes: delta-4 desaturases, delta-5 desaturases, delta-6 desaturases, delta-12 desaturases, delta-15 desaturases, delta-17 desaturases, delta-9 desaturases, delta-8 desaturases, delta-9 elongases, C_{14/16} elongases, C_{16/18} elongases, C_{18/20} elongases and C_{20/22} elongases, although it is to be recognized that the specific enzymes (and genes encoding those enzymes) introduced and the specific PUFAs produced are by no means limiting to the invention herein.

TABLE 3
Lipid Profiles of Representative Yarrowia lipolytica Strains Engineered to Produce Omega-3/Omega-6 PUFAs

| | | ATCC | Fatty Acid Content (As A Percent [%] of Total Fatty Acids) | | | | | | | | | | | | | | TFAs |
|---|---|---|---|---|---|---|---|---|---|---|---|---|---|---|---|---|---|
| | | Deposit | | | | | | 18.3 | | 20:2 | | | | | DPA | | % |
| Strain | Reference | No. | 16:0 | 16:1 | 18:0 | 18:1 | 18:2 | (ALA) | GLA | (EDA) | DGLA | ARA | ETA | EPA | n-3 | DHA | DCW |
| Wildtype | U.S. | #76982 | 14 | 11 | 3.5 | 34.8 | 31 | 0 | 0 | — | — | — | — | — | — | — | — |
| pDMW208 | Pat. No. | — | 11.9 | 8.6 | 1.5 | 24.4 | 17.8 | 0 | 25.9 | — | — | — | — | — | — | — | — |
| pDMW208- | 7,465,564 | — | 16.2 | 1.5 | 0.1 | 17.8 | 22.2 | 0 | 34 | — | — | — | — | — | — | — | — |
| D62 | | | | | | | | | | | | | | | | | |
| M4 | U.S. Pat. | — | 15 | 4 | 2 | 5 | 27 | 0 | 35 | — | 8 | 0 | 0 | 0 | — | — | — |
| | Appl. Pub. | | | | | | | | | | | | | | | | |
| | No. 2006- | | | | | | | | | | | | | | | | |
| | 0115881- | | | | | | | | | | | | | | | | |
| | A1 | | | | | | | | | | | | | | | | |
| Y2034 | U.S. Pat. | — | 13.1 | 8.1 | 1.7 | 7.4 | 14.8 | 0 | 25.2 | — | 8.3 | 11.2 | — | — | — | — | — |
| Y2047 | No. | PTA- | 15.9 | 6.6 | 0.7 | 8.9 | 16.6 | 0 | 29.7 | — | 0 | 10.9 | — | — | — | — | — |
| Y2214 | 7,588,931 | 7186 | | | | | | | | | | | | | | | |
| | | — | 7.9 | 15.3 | 0 | 13.7 | 37.5 | 0 | 0 | — | 7.9 | 14 | — | — | — | — | — |
| EU | U.S. Pat. | — | 19 | 10.3 | 2.3 | 15.8 | 12 | 0 | 18.7 | — | 5.7 | 0.2 | 3 | 10.3 | — | — | 36 |
| Y2072 | Appl. Pub. | — | 7.6 | 4.1 | 2.2 | 16.8 | 13.9 | 0 | 27.8 | — | 3.7 | 1.7 | 2.2 | 15 | — | — | — |
| Y2102 | No. 2006- | — | 9 | 3 | 3.5 | 5.6 | 18.6 | 0 | 29.6 | — | 3.8 | 2.8 | 2.3 | 18.4 | — | — | — |
| Y2088 | 0115881- | — | 17 | 4.5 | 3 | 2.5 | 10 | 0 | 20 | — | 3 | 2.8 | 1.7 | 20 | — | — | — |
| Y2089 | A1 | — | 7.9 | 3.4 | 2.5 | 9.9 | 14.3 | 0 | 37.5 | — | 2.5 | 1.8 | 1.6 | 17.6 | — | — | — |
| Y2095 | | — | 13 | 0 | 2.6 | 5.1 | 16 | 0 | 29.1 | — | 3.1 | 1.9 | 2.7 | 19.3 | — | — | — |
| Y2090 | | — | 6 | 1 | 6.1 | 7.7 | 12.6 | 0 | 26.4 | — | 6.7 | 2.4 | 3.6 | 26.6 | — | — | 22.9 |
| Y2096 | | PTA- | 8.1 | 1 | 6.3 | 8.5 | 11.5 | 0 | 25 | — | 5.8 | 2.1 | 2.5 | 28.1 | — | — | 20.8 |
| | | 7184 | | | | | | | | | | | | | | | |
| Y2201 | | PTA- | 11 | 16.1 | 0.7 | 18.4 | 27 | 0 | — | 3.3 | 3.3 | 1 | 3.8 | 9 | — | — | — |
| | | 7185 | | | | | | | | | | | | | | | |
| Y3000 | U.S. Pat. | PTA- | 5.9 | 1.2 | 5.5 | 7.7 | 11.7 | 0 | 30.1 | — | 2.6 | 1.2 | 1.2 | 4.7 | 18.3 | 5.6 | — |
| | No. | 7187 | | | | | | | | | | | | | | | |
| | 7,550,286 | | | | | | | | | | | | | | | | |
| Y4001 | U.S. Pat. | — | 4.3 | 4.4 | 3.9 | 35.9 | 23 | 0 | — | 23.8 | 0 | 0 | 0 | — | — | — | — |
| Y4036 | Appl. Pub. | — | 7.7 | 3.6 | 1.1 | 14.2 | 32.6 | 0 | — | 15.6 | 18.2 | 0 | 0 | — | — | — | — |
| Y4070 | No. 2009- | — | 8 | 5.3 | 3.5 | 14.6 | 42.1 | 0 | — | 6.7 | 2.4 | 11.9 | — | — | — | — | — |
| Y4086 | 0093543- | — | 3.3 | 2.2 | 4.6 | 26.3 | 27.9 | 6.9 | — | 7.6 | 1 | 0 | 2 | 9.8 | — | — | 28.6 |
| Y4128 | A1 | PTA- | 6.6 | 4 | 2 | 8.8 | 19 | 2.1 | — | 4.1 | 3.2 | 0 | 5.7 | 42.1 | — | — | 18.3 |
| | | 8614 | | | | | | | | | | | | | | | |
| Y4158 | | — | 3.2 | 1.2 | 2.7 | 14.5 | 30.4 | 5.3 | — | 6.2 | 3.1 | 0.3 | 3.4 | 20.5 | — | — | 27.3 |
| Y4184 | | — | 3.1 | 1.5 | 1.8 | 8.7 | 31.5 | 4.9 | — | 5.6 | 2.9 | 0.6 | 2.4 | 28.9 | — | — | 23.9 |
| Y4217 | | — | 3.9 | 3.4 | 1.2 | 6.2 | 19 | 2.7 | — | 2.5 | 1.2 | 0.2 | 2.8 | 48.3 | — | — | 20.6 |
| Y4259 | | — | 4.4 | 1.4 | 1.5 | 3.9 | 19.7 | 2.1 | — | 3.5 | 1.9 | 0.6 | 1.8 | 46.1 | — | — | 23.7 |
| Y4305 | | — | 2.8 | 0.7 | 1.3 | 4.9 | 17.6 | 2.3 | — | 3.4 | 2 | 0.6 | 1.7 | 53.2 | — | — | 27.5 |
| Y4127 | Int'l. App. | PTA- | 4.1 | 2.3 | 2.9 | 15.4 | 30.7 | 8.8 | — | 4.5 | 3.0 | 3.0 | 2.8 | 18.1 | — | — | — |
| | Pub. No. | 8802 | | | | | | | | | | | | | | | |
| Y4184 | WO 2008/ | — | 2.2 | 1.1 | 2.6 | 11.6 | 29.8 | 6.6 | — | 6.4 | 2.0 | 0.4 | 1.9 | 28.5 | — | — | 24.8 |
| | 073367 | | | | | | | | | | | | | | | | |
| Y8404 | U.S. Pat. | — | 2.8 | 0.8 | 1.8 | 5.1 | 20.4 | 2.1 | | 2.9 | 2.5 | 0.6 | 2.4 | 51.1 | — | — | 27.3 |
| Y8406 | Appl. Pub. | PTA- | 2.6 | 0.5 | 2.9 | 5.7 | 20.3 | 2.8 | | 2.8 | 2.1 | 0.5 | 2.1 | 51.2 | — | — | 30.7 |
| | No. 2010- | 10025 | | | | | | | | | | | | | | | |
| Y8412 | 0317072- | PTA- | 2.5 | 0.4 | 2.6 | 4.3 | 19.0 | 2.4 | | 2.2 | 2.0 | 0.5 | 1.9 | 55.8 | — | — | 27.0 |
| | A1 | 10026 | | | | | | | | | | | | | | | |
| Y8647 | | — | 1.3 | 0.2 | 2.1 | 4.7 | 20.3 | 1.7 | | 3.3 | 3.6 | 0.7 | 3.0 | 53.6 | — | — | 37.6 |
| Y8649 | | — | 2.4 | 0.3 | 2.9 | 3.7 | 18.8 | 2.2 | | 2.1 | 2.4 | 0.6 | 2.1 | 55.8 | — | — | 27.9 |
| Y8650 | | — | 2.2 | 0.3 | 2.9 | 3.8 | 18.8 | 2.4 | | 2.1 | 2.4 | 0.6 | 2.1 | 56.1 | — | — | 28.2 |
| Y9028 | | — | 1.3 | 0.2 | 2.1 | 4.4 | 19.8 | 1.7 | | 3.2 | 2.5 | 0.8 | 1.9 | 54.5 | — | — | 39.6 |
| Y9031 | | — | 1.3 | 0.3 | 1.8 | 4.7 | 20.1 | 1.7 | | 3.2 | 3.2 | 0.9 | 2.6 | 52.3 | — | — | 38.6 |
| Y9477 | | — | 2.6 | 0.5 | 3.4 | 4.8 | 10.0 | 0.5 | | 2.5 | 3.7 | 1.0 | 2.1 | 61.4 | — | — | 32.6 |
| Y9497 | | — | 2.4 | 0.5 | 3.2 | 4.6 | 11.3 | 0.8 | | 3.1 | 3.6 | 0.9 | 2.3 | 58.7 | — | — | 33.7 |
| Y9502 | | — | 2.5 | 0.5 | 2.9 | 5.0 | 12.7 | 0.9 | | 3.5 | 3.3 | 0.8 | 2.4 | 57.0 | — | — | 37.1 |
| Y9508 | | — | 2.3 | 0.5 | 2.7 | 4.4 | 13.1 | 0.9 | | 2.9 | 3.3 | 0.9 | 2.3 | 58.7 | — | — | 34.9 |
| Y8143 | | — | 4.2 | 1.5 | 1.4 | 3.6 | 18.1 | 2.6 | | 1.7 | 1.6 | 0.6 | 1.6 | 50.3 | — | — | 22.3 |
| Y8145 | | — | 4.3 | 1.7 | 1.4 | 4.8 | 18.6 | 2.8 | | 2.2 | 1.5 | 0.6 | 1.5 | 48.5 | — | — | 23.1 |
| Y8259 | | PTA- | 3.5 | 1.3 | 1.3 | 4.8 | 16.9 | 2.3 | | 1.9 | 1.7 | 0.6 | 1.6 | 53.9 | — | — | 20.5 |
| | | 10027 | | | | | | | | | | | | | | | |
| Y8367 | | — | 3.7 | 1.2 | 1.1 | 3.4 | 14.2 | 1.1 | | 1.5 | 1.7 | 0.8 | 1.0 | 58.3 | — | — | 18.4 |
| Y8370 | | — | 3.4 | 1.1 | 1.4 | 4.0 | 15.7 | 1.9 | | 1.7 | 1.9 | 0.6 | 1.5 | 56.4 | — | — | 23.3 |
| Y8670 | | — | 1.9 | 0.4 | 3.4 | 4.3 | 17.0 | 1.5 | | 2.2 | 1.7 | 0.6 | 1.1 | 60.9 | — | — | 27.3 |
| Y8672 | | — | 2.3 | 0.4 | 2.0 | 4.0 | 16.1 | 1.4 | | 1.8 | 1.6 | 0.7 | 1.1 | 61.8 | — | — | 26.5 |

One of skill in the art will appreciate that the methodology of the present invention is not limited to the use of microbial biomass obtained from the Yarrowia lipolytica strains described above, nor to the species (i.e., Yarrowia lipolytica) or genus (i.e., Yarrowia) in which the invention has been demonstrated, as the means to introduce a PUFA biosynthetic pathway into an oleaginous yeast are well known. Instead, any oleaginous yeast or any other suitable microbe capable of producing microbial oils comprising at least one PUFA will be equally suitable for use in the present methodologies.

A microbial species producing a lipid containing at least one desired PUFA may be cultured and grown in a fermentation medium under conditions whereby the lipid is produced by the microorganism (FIG. 1A, A). Typically, the microorganism is fed with a carbon and nitrogen source, along with a number of additional chemicals or substances that allow growth of the microorganism and/or production of the microbial oil comprising at least one PUFA. The fermentation conditions will depend on the microorganism used, as described in the above citations, and may be optimized for a high content of the at least one PUFA in the resulting microbial biomass.

In general, media conditions may be optimized by modifying the type and amount of carbon source, the type and amount of nitrogen source, the carbon-to-nitrogen ratio, the amount of different mineral ions, the oxygen level, growth temperature, pH, length of the biomass production phase, length of the oil accumulation phase and the time and method of cell harvest. For example, Yarrowia lipolytica are generally grown in a complex media such as yeast extract-peptone-dextrose broth (YPD) or a defined minimal media (e.g., Yeast Nitrogen Base (DIFCO Laboratories, Detroit, Mich.) that lacks a component necessary for growth and thereby forces selection of the desired recombinant expression cassettes that enable PUFA production).

When the desired amount of microbial oil comprising at least one PUFA has been produced by the microorganism, the fermentation medium may be processed to obtain untreated microbial biomass comprising the microbial oil, via drying, de-watering, pelletizing and/or granulating, for example (FIG. 1A, B).

More specifically, for example, the fermentation medium may be filtered or otherwise treated to remove at least part of the aqueous component (e.g., by drying). As will be appreciated by those in the art, the untreated microbial biomass typically includes water. Preferably, a portion of the water is removed from the untreated microbial biomass after microbial fermentation to provide a microbial biomass with a moisture level of less than 10 weight percent, more preferably a moisture level of less than 5 weight percent, and most preferably a moisture level of 3 weight percent or less. The microbial biomass moisture level can be controlled in drying. Preferably, the microbial biomass has a moisture level in the range of about 1 to 10 weight percent.

The fermentation medium and/or the microbial biomass may be pasteurized or treated via other means to reduce the activity of endogenous microbial enzymes that can harm the microbial oil and/or PUFA products.

The untreated microbial biomass is then subjected to at least one disruption step, prior to extraction with a solvent, to produce a “untreated disrupted microbial biomass” (FIG. 1A, C). The disruption may occur by mechanical/physical means (e.g., via bead beaters, screw extrusion, etc.) or by chemical means (e.g., via enzymatic treatment or osmotic treatment to promote cell lysing). This disruption provides greater accessibility to the microbe's cell contents.

The untreated disrupted microbial biomass is then processed, e.g., extracted, with a solvent (FIG. 1A, D) to obtain an extracted oil and residual biomass. The extracted oil comprises a lipid fraction substantially free of PLs (FIG. 1A, I), while the residual biomass comprises PLs (FIG. 1A, E).

Although oil extraction can occur via treatment with various organic solvents (e.g., hexane, iso-hexane), via enzymatic extraction, via osmotic shock, via ultrasonic extraction, via supercritical fluid extraction (e.g., CO₂ extraction), via saponification and via combinations of these methods, in preferred embodiments herein, the solvent comprises liquid or supercritical fluid CO₂.

Supercritical fluids (SCF) exhibit properties intermediate between those of gases and liquids. A key feature of a SCF is that the fluid density can be varied continuously from liquid-like to gas-like densities by varying either the temperature or pressure, or a combination thereof. Various density-dependent physical properties likewise exhibit similar continuous variation in this region. Some of these properties include, but are not limited to, solvent strength (as evidenced by the solubilities of various substances in the SCF media), polarity, viscosity, diffusivity, heat capacity, thermal conductivity, isothermal compressibility, expandability, contractibility, fluidity, and molecular packing. The density variation in a SCF also influences the chemical potential of solutes and hence, reaction rates and equilibrium constants. Thus, the solvent environment in a SCF media can be optimized for a specific application by tuning the various density-dependent fluid properties.

A fluid is in the SCF state when the system temperature and pressure exceed the corresponding critical point values defined by the critical temperature (T_{c}) and pressure (P_{c}). For pure substances, the T_{c} and P_{c} are the highest at which vapor and liquid phases can coexist. Above the T_{c}, a liquid does not form for a pure substance, regardless of the applied pressure. Similarly, the P_{c} and critical molar volume are defined at this T_{c} corresponding to the state at which the vapor and liquid phases merge. Although more complex for multicomponent mixtures, a mixture critical state is similarly identified as the condition at which the properties of coexisting vapor and liquid phases become indistinguishable. For a discussion of supercritical fluids, see Kirk-Othmer Encycl. of Chem. Technology, 4^{th} Ed., Vol. 23, pg. 452-477.

Any suitable SCF or liquid solvent may be used in the primary extraction step, e.g., the processing of the untreated disrupted microbial biomass with a solvent to separate the PUFA-containing microbial oil from the microbial biomass, including, but not limited to, CO₂, tetrafluoromethane, ethane, ethylene, propane, propylene, butane, isobutane, isobutene, pentane, hexane, cyclohexane, benzene, toluene, xylenes, and mixtures thereof, provided that it is inert to all reagents and products. Preferred solvents include CO₂ or a C₃-C₆ alkane. More preferred solvents are CO₂, pentane, butane, and propane. Most preferred solvents are SCF solvents comprising CO₂. The SCF comprising CO₂ may further comprise at least one additional solvent (i.e., a cosolvent), for example one or more of the solvents listed above, as long as the presence or amount of the additional solvent is not deleterious to the process, for example does not solubilize the PLs contained in the microbial biomass during the primary extraction step. However, a polar cosolvent such as ethanol, methanol, acetone, or the like may be added to intentionally impart polarity to the solvent phase to enable extraction of the PLs from the residual microbial biomass (i.e., during optional secondary extractions) to isolate the PLs.

Untreated disrupted microbial biomass comprising microbial oil comprising at least one PUFA may be processed with liquid or supercritical CO₂ under suitable extraction conditions (FIG. 1A, D) to provide an extracted oil and a residual biomass according to at least two methods. According to a first method, processing the untreated disrupted microbial biomass with CO₂ is performed multiple times under extraction/fractionation conditions corresponding to increasing solvent density, for example under increasing pressure and/or decreasing temperature, to obtain at least one extract comprising a refined lipid composition comprising at least one PUFA. Although the refined lipid composition of the extracts varies in the distribution of FFAs, MAGs, DAGs, and TAGs according to their relative solubilities, which depend upon the solvent density corresponding to the selected extraction conditions of each of the multiple extractions, at least one refined lipid composition will be enriched in TAGs (FIG. 1B, L) relative to the oil composition of the untreated disrupted microbial biomass.

Alternatively, and according to the present methods, in a second method the untreated disrupted microbial biomass is processed with a solvent such as CO₂ under extraction conditions (FIG. 1A, D) selected to provide an extracted oil comprising a lipid fraction substantially free of PLs (FIG. 1A, I), which subsequently undergoes a series of multiple staged pressure letdown fractionation steps to provide refined lipid compositions. Each of these staged pressure letdown steps is conducted in a separator vessel at pressure and temperature conditions corresponding to decreasing solvent density to isolate a liquid-phase refined lipid composition which can be separated from the extract phase by, for example, simple decantation. The refined lipid composition(s) which are provided vary in the distribution of FFAs, MAGs, DAGs, and TAGs according to their relative solubilities, which depend upon the solvent density corresponding to the selected conditions of the staged separator vessels. At least one refined lipid composition will be enriched in TAGs (FIG. 1B, L) relative to the oil composition of the untreated disrupted microbial biomass.

The refined lipid compositions obtained by the second method may correspond to the extracts obtained in the first method when extraction conditions are appropriately matched. It is thus believed possible to exemplify the refined lipid compositions obtainable by the present method through performance of the first method.

According to the present methods, the untreated disrupted microbial biomass may be processed with a solvent such as liquid or SCF CO₂ at a temperature and pressure and for a processing time sufficient to obtain an extract (i.e., an extracted oil) comprising a lipid fraction substantially free of PLs (FIG. 1A, I). The lipid fraction may comprise neutral lipids (e.g., TAGs, DAGs, and MAGs) and FFAs. A sufficient processing time, as well as appropriate CO₂ to biomass ratios, may be determined by generating extraction curves for a particular sample of microbial biomass, for example as described in Example 1. These extraction curves are dependent upon the extraction conditions of temperature, pressure, CO₂ flow rate, and variables such as the extent of cell disruption and the form of the biomass. In one embodiment of the present methods, the solvent comprises liquid or supercritical fluid CO₂ and the mass ratio of CO₂ to the microbial biomass is from about 20:1 to about 70:1, for example from about 20:1 to about 50:1.

The extract comprising a lipid fraction substantially free of PLs (FIG. 1A, I) may then optionally be fractionated at least once to obtain a refined lipid composition. The fractionation may be performed by altering the temperature, the pressure, or the temperature and the pressure of the fractionating conditions. Fractionation may be accomplished in one of several separation processes including, for example, a sequential pressure reduction of the SCF-rich extract, liquid or SCF solvent extraction in a series of mixer-settler stages or extraction column, short-path distillation, vacuum steam stripping, or melt crystallization. The step of fractionating the extract may be repeated one or more times to provide additional refined lipid compositions.

Reducing the pressure, for example, of the extract lowers the solubility of the dissolved solutes, forming a separate liquid phase in each separation vessel. The temperature of the extract being fed to each separation vessel can be adjusted, for example through the use of heat exchangers, to provide the desired solvent density and corresponding solute solubility in each separation vessel. The initial extract consists of a complex mixture of various types of lipid components (e.g., FFAs, MAGs, DAGs, and TAGs) which exhibit similar solubility parameters, so an exact separation of the various components will not be achieved, but rather each refined lipid composition obtained in the fractionation step will contain a distribution of products. However, in general, the less soluble compounds condense in the first separation vessel operating at the highest pressure, and the most soluble compounds condense in the final separation vessel operating at the lowest pressure. The final separation vessel reduces the pressure of the extract phase sufficiently to essentially remove the bulk of the remaining solute in the extract phase, and the relatively pure CO₂ stream from the top of this vessel may be recycled back to the initial extraction vessel(s).

The refined lipid composition(s) comprising at least one PUFA is substantially free of PLs. At least one of the refined lipid compositions will be enriched in TAGs relative to the oil composition of the microbial biomass (FIG. 1B, L) and may further comprise DAGs, MAGs, or combinations thereof. The refined lipid composition enriched in TAGs may further comprise FFAs. Other refined lipid compositions which may be obtained separately or in combination in the fractionation step include a TAG enriched product that is depleted in FFAs, a FFA enriched product that is depleted in TAGs, a FFA enriched product that is enriched in MAGs and/or DAGs, a FFA enriched product that is depleted in MAGs and/or DAGs, a TAG enriched product that is enriched in MAGs and/or DAGs, and a TAG enriched product that is depleted in MAGs and/or DAGs (FIG. 1B, K). According to the fractionating conditions employed, in one embodiment of the present methods, the at least one refined lipid composition enriched in TAGs may be depleted in FFAs relative to the oil composition of the microbial biomass. In one embodiment, the at least one refined lipid composition enriched in TAGs may be enriched in at least one PUFA relative to the oil composition of the microbial biomass.

In one embodiment, the at least one refined lipid composition enriched in TAGs may be enriched in at least one PUFA having 20 or more carbon atoms relative to the oil composition of the biomass, wherein the at least one PUFA having 20 or more carbon atoms may preferably comprise at least four carbon-carbon double bonds.

The processing and fractionating temperatures may be chosen to provide liquid or SCF CO₂, to be within the thermal stability range of the at least one PUFA, and to provide sufficient density of the CO₂ to solubilize the TAGs, DAGs, MAGs, and FFAs. Generally, the processing and fractionating temperatures may be from about 20° C. to about 100° C., for example from about 35° C. to about 100° C.; the pressure may be from about 60 bar to about 800 bar, for example from about 80 bar to about 600 bar.

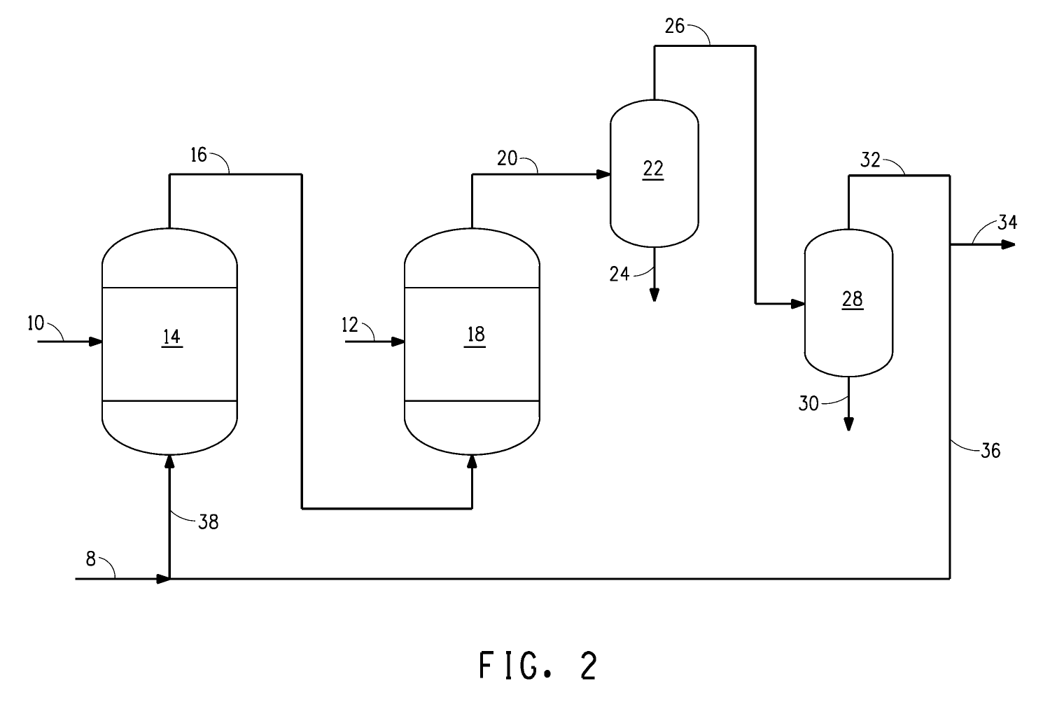

FIG. 2 schematically illustrates one embodiment of the methods of the invention. In FIG. 2, stream 10 comprising untreated disrupted microbial biomass and stream 38 comprising CO₂ are shown entering vessel 14. Stream 12 comprising untreated disrupted microbial biomass and stream 16 comprising a mixture of equilibrated CO₂ and extract are shown entering vessel 18. Processing of the untreated disrupted microbial biomass comprising microbial oil comprising at least one PUFA with CO₂ occurs in vessel 14 at an initial temperature T₁₄ and pressure P₁₄, and in vessel 18 at a temperature T₁₈ and pressure P₁₈. T₁₄ may be the same as or different from T₁₈; P₁₄ may be the same as or different from P₁₈. The resulting mixture of equilibrated CO₂ and extract leaves vessel 14 as stream 16 to enter vessel 18, in which further processing of the microbial biomass and the CO₂ occurs to provide an extract comprising a lipid fraction substantially free of PLs, shown as stream 20. The residual biomass (not shown) remains in vessels 14 and 16. Additional extraction vessels may be included in the process, if desired (not shown). Alternatively, the process may use only one extraction vessel if desired (not shown). The use of more than one extraction vessel may be advantageous as this can enable continuous CO₂ flow through the process by changing the relative order of solvent addition to the extraction vessels (not shown) and while one or more extraction vessels are taken off line (not shown), for example to charge microbial biomass or to remove residual biomass.

Downstream of the extraction vessels are shown two separation vessels arranged in series, vessels 22 and 28, in which fractionation of the extract is performed through a staged pressure reduction, optionally with adjustment of the temperature, for example through the use of heat exchangers (not shown). Additional separation vessels could be included in the process, if desired (not shown). The extract comprising CO₂ and a lipid fraction substantially free of PLs is shown entering vessel 22 as stream 20. In vessel 22, the pressure P₂₂ is lower than P₁₈ and the temperature T₂₂ may be the same as or different from T₁₈; under the operating conditions of the process, a separate liquid phase comprising the less soluble lipid components is formed. The separate liquid phase resulting from fractionation of the extract is shown leaving vessel 22 as stream 24, which represents a first refined lipid composition obtained by the present method. The remaining extract, shown as stream 26, is introduced to the next separation vessel 28, where the pressure P₂₈ is reduced compared to P₂₂ and the temperature T₂₈ may or may not be the same as T₂₂. The operating conditions of the process enable formation of a separate liquid phase in vessel 28, which is shown leaving separation vessel 28 as stream 30. Stream 30 represents a second refined lipid composition obtained by the present method.

From vessel 28, the remaining extract comprising relatively pure CO₂, shown as stream 32, may be recycled to extraction vessel 14 and/or to another extraction vessel (not shown). Recycling the CO₂ typically provides economic benefits over once-through CO₂ usage. A purge stream, shown as stream 34, can be used to remove volatile components which may build up with continuous recycle of the CO₂ to the process. Make-up CO₂ may be added to offset the CO₂ loss incurred through a purge. Make-up CO₂ may be added to the recycle CO₂ stream as shown in FIG. 2 by make-up CO₂ stream 8 joining stream 36 to provide the combined CO₂ stream 38. Alternatively, additional CO₂ could be added to vessel 14 and/or vessel 18 as a separate feed stream (not shown).

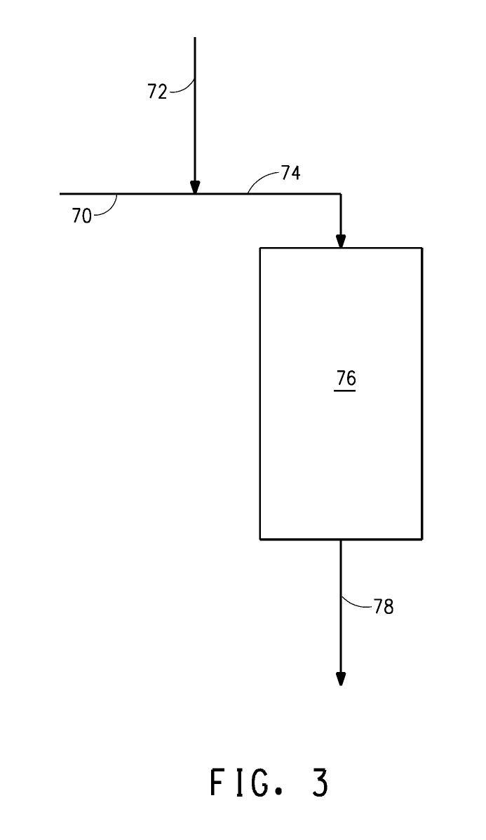

FIG. 3 schematically illustrates one embodiment of the extraction step of the method of the invention. In FIG. 3, stream 70 comprising CO₂ is introduced into extraction vessel 76, which contains untreated disrupted microbial biomass (not shown). Optionally, a cosolvent (shown as stream 72) is added to the CO₂ stream using a pump (not shown) to provide the combined stream 74 comprising CO₂ and cosolvent. In the case where a cosolvent is not used, stream 70 and stream 74 are the same and contain only CO₂. Processing the CO₂ with the untreated disrupted microbial biomass comprising at least one PUFA occurs in vessel 76, and the extract comprising a lipid fraction substantially free of PLs is removed from the vessel as stream 78 along with the CO₂ solvent and optionally the cosolvent. The residual biomass (not shown) remains in the extraction vessel. The extract comprising a lipid fraction substantially free of PLs may then be fractionated in at least one separation vessel, as described above in reference to FIG. 2, or optionally, the lipid fraction substantially free of PLs may be isolated from the extract by venting the CO₂ and optionally the cosolvent (not shown).

The residual biomass from the above primary extraction comprises PLs (FIG. 1A, E). This residual biomass may be extracted a second time with a polar extraction solvent, (FIG. 1B, F) for example a polar organic solvent such as methylene chloride or a mixed solvent comprising CO₂ and a polar cosolvent such as an alcohol, to obtain a PL fraction substantially free of neutral lipids (i.e., a “residual biomass extract consisting essentially of PLs”; FIG. 1B, H). The polar cosolvent may comprise methanol, ethanol, 1-propanol, and/or 2-propanol, for example. Either the residual biomass comprising PLs (FIG. 1A, E) or the extracted PL fraction (FIG. 1B, H) may be suitable for use as, e.g., an aquaculture feed.

The CO₂-based extraction/fractionation process described herein offers several advantages relative to conventional organic solvent-based processes. For example, CO₂ is nontoxic, nonflammable, environmentally friendly, readily available, and inexpensive. CO₂ (T_{c}=31.1° C.) can extract thermally labile lipids from microbial biomass at relatively low temperatures to minimize lipid degradation in the microbial oil. The extracted lipids may be isolated from the CO₂ solvent by simply venting the CO₂ from the pressurized extract rather than through thermal processing to strip organic solvents. The lipid fraction in the extract is substantially free of PLs and may be isolated from the microbial biomass. The residual microbial biomass containing PLs (FIG. 1A, E) may be a saleable co-product, for example, for aquaculture feed. The PLs may be extracted from the residual microbial biomass as a relatively pure co-product depleted in neutral lipids (FIG. 1B, H). The extracted lipid fraction substantially free of PLs (FIG. 1A, I) may be fractionated (FIG. 1B, J) to produce, for example, a refined lipid composition enriched in FFAs and DAGs (and depleted in TAGs) (FIG. 1B, K) relative to the disrupted microbial biomass and a refined lipid composition enriched in TAGs (and depleted in FFAs and DAGs) (FIG. 1B, L) relative to the disrupted microbial biomass.

Thus, methods for obtaining a refined lipid composition comprising at least one PUFA are provided, wherein:

a) an untreated disrupted microbial biomass having an oil composition comprising at least one PUFA is processed with a solvent comprising liquid or SCF CO₂ to obtain:
(i) an extract comprising a lipid fraction substantially free of PLs; and,
(ii) a residual biomass comprising PLs; and,

b) the extract obtained in step (a), part (i) is fractionated at least once to obtain a refined lipid composition comprising at least one PUFA, wherein the refined lipid composition is enriched in TAGs relative to the oil composition of the untreated disrupted microbial biomass.

Preferably, the processing of step (a) is done at a temperature from about 20° C. to about 100° C. and at a pressure from about 60 bar to about 800 bar and the fractionating of step (b) is done at a temperature from about 35° C. to about 100° C. and at a pressure from about 80 bar to about 600 bar. The methods disclosed herein may be performed by altering the temperature, the pressure, or the temperature and the pressure, of the fractionating conditions.

In still another aspect, the methods disclosed herein also further comprise a step selected from the group consisting of:
(1) fractionating the extract obtained in step (a), part (i) to obtain a refined lipid composition comprising at least one PUFA, wherein the refined lipid composition is enriched in lipid components selected from the group consisting of DAGs, MAGs, FFAs and combinations thereof relative to the oil composition of the untreated disrupted microbial biomass; and,
(2) processing the residual biomass comprising PLs of step (a), part (ii) with an extractant to obtain a residual biomass extract consisting essentially of PLs.

In another embodiment, the methods disclosed herein utilize untreated disrupted microbial biomass comprising oleaginous microbial cells. These oleaginous microbial cells preferably are selected from the group consisting of yeast, algae, euglenoids, stramenopiles, fungi, and mixtures thereof. More preferably, the cells are a member of a genus selected from the group consisting of Mortierella, Thraustochytrium, Schizochytrium, Yarrowia, Candida, Rhodotorula, Rhodosporidium, Cryptococcus, Trichosporon, and Lipomyces, wherein the genus Yarrowia is particularly preferred.

The microbial biomass will comprise at least one PUFA selected from the group consisting of LA, GLA, EDA, DGLA, ARA, DTA, DPAn-6, ALA, STA, ETrA, ETA, EPA, DPAn-3, DHA, and mixtures thereof. Preferably, the at least one PUFA is selected from the group consisting of EDA, DGLA, ARA, DTA, DPAn-6, ETrA, ETA, EPA, DPAn-3, DHA, and mixtures thereof (i.e., corresponding to PUFAs having at least twenty carbon atoms). As demonstrated in the present examples, the untreated disrupted microbial biomass will preferably comprise at least 25 wt % of EPA measured as a wt % of TFAs, although this should not be construed as limiting to the invention herein.

In alternate embodiments, provided herein is a method comprising processing an untreated disrupted microbial biomass having an oil composition comprising at least one PUFA with a solvent comprising liquid or SCF CO₂ to obtain:
(i) an extract comprising a lipid fraction substantially free of PLs; and,
(ii) a residual biomass comprising PLs;

wherein said untreated disrupted microbial biomass is obtained from an oleaginous microorganism of the genus Yarrowia that accumulates in excess of 25% of its dry cell weight as oil; and,

wherein said oil composition comprising at least one PUFA comprises at least 25 weight percent of a PUFA having at least twenty carbon atoms and four or more carbon-carbon double bonds, measured as a weight percent of TFAs.

The untreated disrupted microbial biomass is preferably obtained from Yarrowia lipolytica and the at least one PUFA preferably comprises EPA.

Extracted oil compositions and/or refined lipid compositions comprising at least one PUFA, such as EPA (or derivatives thereof), will have well known clinical and pharmaceutical value. See, e,g., U.S. Pat. Appl. Pub. No. 2009-0093543 A1. For example, lipid compositions comprising PUFAs may be used as dietary substitutes, or supplements, particularly infant formulas, for patients undergoing intravenous feeding or for preventing or treating malnutrition. Alternatively, the purified PUFAs (or derivatives thereof) may be incorporated into cooking oils, fats or margarines formulated so that in normal use the recipient would receive the desired amount for dietary supplementation. The PUFAs may also be incorporated into infant formulas, nutritional supplements or other food products and may find use as anti-inflammatory or cholesterol lowering agents. Optionally, the compositions may be used for pharmaceutical use, either human or veterinary.

Supplementation of humans or animals with PUFAs can result in increased levels of the added PUFAs, as well as their metabolic progeny. For example, treatment with EPA can result not only in increased levels of EPA, but also downstream products of EPA such as eicosanoids (i.e., prostaglandins, leukotrienes, thromboxanes), DPAn-3 and DHA. Complex regulatory mechanisms can make it desirable to combine various PUFAs, or add different conjugates of PUFAs, in order to prevent, control or overcome such mechanisms to achieve the desired levels of specific PUFAs in an individual.

Alternatively, PUFAs, or derivatives thereof, can be utilized in the synthesis of animal and aquaculture feeds, such as dry feeds, semi-moist and wet feeds, since these formulations generally require at least 1-2% of the nutrient composition to be omega-3 and/or omega-6 PUFAs (U.S. Pat. App. Pub No. 2006-0115881-A1). In particular, it is contemplated herein that the residual biomass comprising PLs may be suitable for use as an aquaculture feed or component thereof. Alternately, the residual biomass may be extracted with an extractant to obtain a residual biomass extract consisting essentially of PLs, which may be useful in the preparation of aquaculture feeds.

### EXAMPLES

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions. Reference should be made to the appended claims, rather than to the foregoing specification, as indicating the scope of the invention.

The following abbreviations are used:

“HPLC” is High Performance Liquid Chromatography, “C” is Celsius, “kPa” is kiloPascal, “mm” is millimeter, “μm” is micrometer, “μL” is microliter, “mL” is milliliter, “L” is liter, “min” is minute, “mM” is millimolar, “cm” is centimeter, “g” is gram, “wt” is weight, “hr” is hour, “temp” or “T” is temperature, “SS” is stainless steel, “in” is inch, “i.d.” is inside diameter, “o.d.” is outside diameter, and “%” is percent.

### Materials

The following materials were used in the examples. All commercial reagents were used as received. All solvents used were HPLC grade. Acetyl chloride was 99+%. TLC plates and solvents were obtained from VWR (West Chester, Pa.). HPLC or SCF grade carbon dioxide was obtained from MG Industries (Malvern, Pa.).

### Microbial Biomass Preparation

Described below are several strains of Yarrowia lipolytica yeast producing various amounts of microbial oil comprising at least one PUFA. Microbial biomass was obtained in a 2-stage fed-batch fermentation process, and then subjected to downstream processing, as described below.

Yarrowia lipolytica Strains: The Comparative Example and Examples 1, 2, 3, 4, 7, 8 and 9 herein utilized Yarrowia lipolytica strain Y8672 biomass. The generation of strain Y8672 is described in U.S. Pat. Appl. Pub. No. 2010-0317072-A1 [hereby incorporated herein by reference]. Strain Y8672, derived from Yarrowia lipolytica ATCC #20362, was capable of producing about 61.8% EPA relative to the total lipids via expression of a delta-9 elongase/delta-8 desaturase pathway.

The final genotype of strain Y8672 with respect to wild type Yarrowia lipolytica ATCC #20362 was Ura+, Pex3−, unknown 1−, unknown 2−, unknown 3−, unknown 4−, unknown 5−, unknown 6−, unknown 7−, unknown 8−, Leu+, Lys+, YAT1::ME3S::Pex16, GPD::ME3S::Pex20, GPD::FmD12::Pex20, YAT1::FmD12::Oct, EXP1::FmD12S::ACO, GPAT::EgD9e::Lip2, FBAINm::EgD9eS::Lip2, EXP1::EgD9eS::Lip1, YAT1::EgD9eS::Lip2, FBAINm::EgD8M::Pex20, FBAIN::EgD8M::Lip1, EXP1::EgD8M::Pex16, GPD::EaD8S::Pex16 (2 copies), YAT1::E389D9eS/EgD8M::Lip1, YAT1::EgD9ES/EgD8M::Aco, FBAIN::EgD5SM::Pex20, YAT1::EgD5SM::Aco, GPM::EgD5SM::Oct, EXP1::EgD5M::Pex16, EXP1::EgD5SM::Lip1, YAT1::EaD5SM::Oct, YAT1::PaD17S::Lip1, EXP1::PaD17::Pex16, FBAINm::PaD17::Aco, GPD::YICPT1::Aco, and YAT1::MCS::Lip1. The structure of the above expression cassettes are represented by a simple notation system of “X::Y::Z”, wherein X describes the promoter fragment, Y describes the gene fragment, and Z describes the terminator fragment, which are all operably linked to one another. Abbreviations are as follows: FmD12 is a Fusarium moniliforme delta-12 desaturase gene [U.S. Pat. No. 7,504,259]; FmD12S is a codon-optimized delta-12 desaturase gene, derived from Fusarium moniliforme [U.S. Pat. No. 7,504,259]; ME3S is a codon-optimized C_{16/18} elongase gene, derived from Mortierella alpina [U.S. Pat. No. 7,470,532]; EgD9e is a Euglena gracilis delta-9 elongase gene [U.S. Pat. No. 7,645,604]; EgD9eS is a codon-optimized delta-9 elongase gene, derived from Euglena gracilis [U.S. Pat. No. 7,645,604]; EgD8M is a synthetic mutant delta-8 desaturase gene [U.S. Pat. No. 7,709,239], derived from Euglena gracilis [U.S. Pat. No. 7,256,033]; EaD8S is a codon-optimized delta-8 desaturase gene, derived from Euglena anabaena [U.S. Pat. No. 7,790,156]; E389D9eS/EgD8M is a DGLA synthase created by linking a codon-optimized delta-9 elongase gene (“E389D9eS”), derived from Eutreptiella sp. CCMP389 delta-9 elongase (U.S. Pat. No. 7,645,604) to the delta-8 desaturase “EgD8M” (supra) [U.S. Pat. Appl. Pub. No. 2008-0254191-A1]; EgD9ES/EgD8M is a DGLA synthase created by linking the delta-9 elongase “EgD9eS” (supra) to the delta-8 desaturase “EgD8M” (supra) [U.S. Pat. Appl. Pub. No. 2008-0254191-A1]; EgD5M and EgD5SM are synthetic mutant delta-5 desaturase genes [U.S. Pat. App. Pub. 2010-0075386-A1], derived from Euglena gracilis [U.S. Pat. No. 7,678,560]; EaD5SM is a synthetic mutant delta-5 desaturase gene [U.S. Pat. App. Pub. 2010-0075386-A1], derived from Euglena anabaena [U.S. Pat. Appl. Pub. No. 2008-0274521-A1]; PaD17 is a Pythium aphanidermatum delta-17 desaturase gene [U.S. Pat. No. 7,556,949]; PaD17S is a codon-optimized delta-17 desaturase gene, derived from Pythium aphanidermatum [U.S. Pat. No. 7,556,949]; YICPT1 is a Yarrowia lipolytica diacylglycerol cholinephosphotransferase gene [Int'l. App. Pub. No. WO 2006/052870]; and, MCS is a codon-optimized malonyl-CoA synthetase gene, derived from Rhizobium leguminosarum bv. viciae 3841 [U.S. Pat. App. Pub. No. 2010-0159558-A1].

For a detailed analysis of the total lipid content and composition in strain Y8672, a flask assay was conducted wherein cells were grown in 2 stages for a total of 7 days. Based on analyses, strain Y8672 produced 3.3 g/L dry cell weight [“DCW”], total lipid content of the cells was 26.5 [“TFAs % DCW”], the EPA content as a percent of the dry cell weight [“EPA % DCW”] was 16.4, and the lipid profile was as follows, wherein the concentration of each fatty acid is as a weight percent of TFAs [“% TFAs”]: 16:0 (palmitate)—2.3, 16:1 (palmitoleic acid)—0.4, 18:0 (stearic acid)—2.0, 18:1 (oleic acid)—4.0, 18:2 (LA)—16.1, ALA—1.4, EDA—1.8, DGLA—1.6, ARA—0.7, ETrA—0.4, ETA—1.1, EPA—61.8, other—6.4.

Example 6 herein utilized Yarrowia lipolytica strain Y9502 biomass. The generation of strain Y9502 is described in U.S. Pat. Appl. Pub. No. 2010-0317072-A1, hereby incorporated herein by reference. Strain Y9502, derived from Yarrowia lipolytica ATCC #20362, was capable of producing about 57.0% EPA relative to the total lipids via expression of a delta-9 elongase/delta-8 desaturase pathway.

The final genotype of strain Y9502 with respect to wildtype Yarrowia lipolytica ATCC #20362 was Ura+, Pex3−, unknown 1−, unknown 2−, unknown 3−, unknown 4−, unknown 5−, unknown 6−, unknown 7−, unknown 8−, unknown 9−, unknown 10−, YAT1::ME3S::Pex16, GPD::ME3S::Pex20, YAT1::ME3S::Lip1, FBAINm::EgD9eS::Lip2, EXP1::EgD9eS::Lip1, GPAT::EgD9e::Lip2, YAT1::EgD9eS::Lip2, FBAINm::EgD8M::Pex20, EXP1::EgD8M::Pex16, FBAIN::EgD8M::Lip1, GPD::EaD8S::Pex16 (2 copies), YAT1::E389D9eS/EgD8M::Lip1, YAT1::EgD9eS/EgD8M::Aco, FBAINm::EaD9eS/EaD8S::Lip2, GPD::FmD12::Pex20, YAT1::FmD12::Oct, EXP1::FmD12S::Aco, GPDIN::FmD12::Pex16, EXP1::EgD5M::Pex16, FBAIN::EgD5SM::Pex20, GPDIN::EgD5SM::Aco, GPM::EgD5SM::Oct, EXP1::EgD5SM::Lip1, YAT1::EaD5SM::Oct, FBAINm::PaD17::Aco, EXP1::PaD17::Pex16, YAT1::PaD17S::Lip1, YAT1::YICPT::Aco, YAT1::MCS::Lip1, FBA::MCS::Lip1, YAT1::MaLPAAT1S::Pex16. Abbreviations not previously defined are as follows: EaD9eS/EgD8M is a DGLA synthase created by linking a codon-optimized delta-9 elongase gene (“EaD9eS”), derived from Euglena anabaena delta-9 elongase [U.S. Pat. No. 7,794,701] to the delta-8 desaturase “EgD8M” (supra) [U.S. Pat. Appl. Pub. No. 2008-0254191-A1]; and, MaLPAAT1S is a codon-optimized lysophosphatidic acid acyltransferase gene, derived from Mortierella alpina [U.S. Pat. No. 7,879,591].

For a detailed analysis of the total lipid content and composition in strain Y9502, a flask assay was conducted wherein cells were grown in 2 stages for a total of 7 days. Based on analyses, strain Y9502 produced 3.8 g/L dry cell weight [“DCW”], total lipid content of the cells was 37.1 [“TFAs % DCW”], the EPA content as a percent of the dry cell weight [“EPA % DCW”] was 21.3, and the lipid profile was as follows, wherein the concentration of each fatty acid is as a weight percent of TFAs [“% TFAs”]: 16:0 (palmitate)—2.5, 16:1 (palmitoleic acid)—0.5, 18:0 (stearic acid)—2.9, 18:1 (oleic acid)—5.0, 18:2 (LA)—12.7, ALA—0.9, EDA—3.5, DGLA—3.3, ARA—0.8, ETrA—0.7, ETA—2.4, EPA—57.0, other—7.5.

Example 5 herein utilized Yarrowia lipolytica strain Y4305F1B1 biomass. The generation of strain Y4305F1B1, derived from Yarrowia lipolytica ATCC #20362 and capable of producing about 50-52% EPA relative to the total lipids with 28-32% total lipid content [“TFAs % DCW”] via expression of a delta-9 elongase/delta-8 desaturase pathway, is set forth below. Specifically, strain Y4305F1B1 is derived from Yarrowia lipolytica strain Y4305, which has been previously described in the General Methods of U.S. Pat. App. Pub. No. 2008-0254191, published on Apr. 9, 2009, the disclosure of which is hereby incorporated in its entirety. The final genotype of strain Y4305 with respect to wild type Yarrowia lipolytica ATCC #20362 was SCP2− (YALI0E01298g), YALI0C18711g—, Pex10−, YALI0F24167g−, unknown 1−, unknown 3−, unknown 8−, GPD::FmD12::Pex20, YAT1::FmD12::OCT, GPM/FBAIN::FmD12S::OCT, EXP1::FmD12S::Aco, YAT1::FmD12S::Lip2, YAT1::ME3S::Pex16, EXP1::ME3S::Pex20 (3 copies), GPAT::EgD9e::Lip2, EXP1::EgD9eS::Lip1, FBAINm::EgD9eS::Lip2, FBA::EgD9eS::Pex20, GPD::EgD9eS::Lip2, YAT1::EgD9eS::Lip2, YAT1::E389D9eS::OCT, FBAINm::EgD8M::Pex20, FBAIN::EgD8M::Lip1 (2 copies), EXP1::EgD8M::Pex16, GPDIN::EgD8M::Lip1, YAT1::EgD8M::Aco, FBAIN::EgD5::Aco, EXP1::EgD5S::Pex20, YAT1::EgD5S::Aco, EXP1::EgD5S::ACO, YAT1::RD5S::OCT, YAT1::PaD17S::Lip1, EXP1::PaD17::Pex16, FBAINm::PaD17::Aco, YAT1::YICPT1::ACO, GPD::YICPT1::ACO. Abbreviations not previously defined are as follows: E389D9eS is a codon-optimized delta-9 elongase gene, derived from Eutreptiella sp. CCMP389 [U.S. Pat. No. 7,645,604]; EgD5 is a Euglena gracilis delta-5 desaturase [U.S. Pat. No. 7,678,560]; EgD5S is a codon-optimized delta-5 desaturase gene, derived from Euglena gracilis [U.S. Pat. No. 7,678,560]; and, RD5S is a codon-optimized delta-5 desaturase, derived from Peridinium sp. CCMP626 [U.S. Pat. No. 7,695,950].

Total lipid content of the Y4305 cells was 27.5 [“TFAs % DCW”], and the lipid profile was as follows, wherein the concentration of each fatty acid is as a weight percent of TFAs [“% TFAs”]: 16:0 (palmitate)—2.8, 16:1 (palmitoleic acid)—0.7, 18:0 (stearic acid)—1.3, 18:1 (oleic acid)—4.9, 18:2 (LA)—17.6, ALA—2.3, EDA—3.4, DGLA—2.0, ARA—0.6, ETA—1.7 and EPA—53.2.

Strain Y4305 was subjected to transformation with a dominant, non-antibiotic marker for Yarrowia lipolytica based on sulfonylurea [“SU^{R}”] resistance. More specifically, the marker gene is a native acetohydroxyacid synthase (“AHAS” or acetolactate synthase; E.C. 4.1.3.18) that has a single amino acid change, i.e., W497L, that confers sulfonyl urea herbicide resistance (SEQ ID NO:292 of Intl. App. Pub. No. WO 2006/052870).

The random integration of the SU^{R} genetic marker into Yarrowia strain Y4305 was used to identify those cells having increased lipid content when grown under oleaginous conditions relative to the parent Y4305 strain, as described in U.S. Pat. App. Pub. No. 2011-0059204-A1.

When evaluated under 2 liter fermentation conditions, average EPA productivity [“EPA % DCW”] for strain Y4305 was 50-56, as compared to 50-52 for mutant SU^{R} strain Y4305-F1B1. Average lipid content [“TFAs DCW”] for strain Y4305 was 20-25, as compared to 28-32 for strain Y4305-F1B1. Thus, lipid content was increased 29-38% in strain Y4503-F1B1, with minimal impact upon EPA productivity.

Fermentation: Inocula were prepared from frozen cultures of Yarrowia lipolytica in a shake flask. After an incubation period, the culture was used to inoculate a seed fermentor. When the seed culture reached an appropriate target cell density, it was then used to inoculate a larger fermentor. The fermentation is a 2-stage fed-batch process. In the first stage, the yeast were cultured under conditions that promote rapid growth to a high cell density; the culture medium comprised glucose, various nitrogen sources, trace metals and vitamins. In the second stage, the yeast were starved for nitrogen and continuously fed glucose to promote lipid and PUFA accumulation. Process variables including temperature (controlled between 30-32° C.), pH (controlled between 5-7), dissolved oxygen concentration and glucose concentration were monitored and controlled per standard operating conditions to ensure consistent process performance and final PUFA oil quality.

One of skill in the art of fermentation will know that variability will occur in the oil profile of a specific Yarrowia strain, depending on the fermentation run itself, media conditions, process parameters, scale-up, etc., as well as the particular time-point in which the culture is sampled (see, e.g., U.S. Pat. Appl. Pub. No. 2009-0093543-A1).

Downstream Processing: Antioxidants were optionally added to the fermentation broth prior to processing to ensure the oxidative stability of the microbial oil. The yeast microbial biomass was dewatered and washed to remove salts and residual medium, and to minimize lipase activity. Either drum-drying (typically with 80 psig steam) or spray-drying was then performed, to reduce moisture content to less than 5% to ensure oil stability during short term storage and transportation. The drum dried flakes or spray dried powder was mechanically disrupted using a twin-screw extruder to make microbial oil more readily exposed and thereby facilitate extraction.

### General Methods

Method For Determining Lipid Distribution Within Microbial Biomass, Extracted Oil And Residual Biomass Samples: Samples of yeast microbial biomass and residual biomass (i.e., after extraction with CO₂) were extracted using a modification of the method of Bligh & Dyer (based on procedures outlined in Lipid Analysis, 3^{rd} ed., W. W. Christie, Ed., Oily Press: Bridgwater, 2003), separated with thin-layer chromatography (TLC) and directly esterified/transesterified using methanolic hydrogen chloride. Oil samples were dissolved in chloroform/methanol, then separated with TLC and directly esterified/transesterified. The esterified/transesterified samples were analyzed by gas chromatography.

Samples of yeast microbial biomass and residual biomass were typically received as a dry powder. A predetermined portion (100-200 mg or less, depending on the PUFA concentration) of the sample was weighed into a 13×100 mm glass test tube with a Teflon™ cap to which 3 mL volume of a 2:1 (volume:volume) methanol/chloroform solution was added. The sample was vortexed thoroughly and incubated at room temperature for one hr with gentle agitation and inversion. After the hr, 1 mL of chloroform and 1.8 mL of deionized water were added, the mixture was agitated and then centrifuged to separate the two layers that formed. Using a pasteur pipette, the bottom layer was removed into a second, tared 13 mm glass vial and the aqueous top layer was re-extracted with a second 1 mL portion of chloroform for 30 min. The two extracts were combined and considered as the “first extract”. The solvent was removed using a TurboVap™ at 50° C. with dry nitrogen and the remaining oil was resuspended in the appropriate amount of 6:1 (volume:volume) chloroform/methanol to obtain a 100 mg/mL solution.

The extracted oil obtained as described above (for yeast microbial biomass and residual biomass samples) and the oil samples from CO₂ extraction of the microbial biomass were analyzed by thin layer chromatography (TLC). The TLC was typically done using one tank, although a two tank procedure was also employed when individual PLs were to be identified. In the one tank TLC procedure, a 5×20 cm silica gel 60 plate (EMD #5724-3, obtained from VWR) was prepared by drawing a light pencil line all the way across the plate 2 cm from the bottom. An appropriate amount of sample, (˜60 μL) was spotted completely across the plate on top of the pencil line without leaving any space between the spots. A second plate was spotted with known standards and the sample using 1-2 μL amounts. The plates were air dried for 5-10 min and developed using a hexane-diethyl ether-acetic acid mixture (70:30:1 by volume) that had been equilibrated in the tank for at least 30 min with a piece of blotting paper prior to running the plate.

After the plates had been developed to within a ¼ inch of the top, they were dried in a N₂ environment for 15 min. The second plate, with the standards and small sample spot, was then developed in a tank that had been saturated with iodine crystals to serve as a reference for the preparative plate. The bands on the preparative plate were identified by very lightly staining the edge of the bands with iodine and, using a pencil, grouping the bands according to each fraction (i.e., the PL, FFA, TAG and DAG fractions, respectively). The DAG band can show some separation between the 1,2-DAGs, the 1,3-DAGs, and the MAG band, and typically this entire area was cut out as the DAG band. The bands were cut out of the gel and transferred to a 13 mm glass vial. The remainder of the plate was developed in the iodine tank to verify complete removal of the bands of interest.

To the glass vial containing each band, an appropriate amount of triglyceride internal standard in toluene was added. Depending on the visible concentration of each band, 100 μL of a 0.1 to 5 mg/mL internal standard was usually used. A co-solvent (in this case, toluene) was added with the internal standard. If an internal standard was not used, additional co-solvent was added to complete the esterification/trans-esterification of the longer chain lipids. 1 mL of a 1% methanolic hydrogen chloride solution (prepared by slowly adding 5 mL acetyl chloride to 50 mL cooled, dry methanol) was added, the sample capped, gently mixed, and placed in a heating block at 80° C. After one hr, the sample was removed and allowed to cool. 1 mL of a 1 N sodium chloride solution and 400 μL of hexane were added, the sample then vortexed for at least 12 sec and centrifuged to separate the two layers. The top layer was then removed, with care being taken to not contaminate it with any of the aqueous (bottom) layer. The top layer was placed into a GC vial fitted with an insert and capped.

The sample was analyzed using an Agilent Model 6890 Gas Chromatograph (Agilent Technologies, Santa Clara, Calif.), equipped with a Flame Ionization Detector (FID) and an Omegawax 320 column (30 m×0.32 mm ID×25 μm film thickness and manufactured by Supelco (Bellfonte, Pa.)). The helium carrier gas was kept constant within a range of 1-3 mL/min with a split ratio of 20:1 or 30:1. The oven conditions were as follows: initial temperature of 160° C. with an initial time of 0 min and an equilibration time of 0.5 min. The temperature ramps were 5 degrees/min to 200° C. for a final hold time of 0 min then 10 degrees/min to 240° C. for four min of hold time for a total of 16 min. The inlet was set to 260° C. The FID detector was also set to 260° C. A Nu-Chek Prep GLC reference standard (#461) was run for retention time verification.

The GC results were collected using Agilent's Custom Reports and the area of each fatty acid was transferred to an Excel spreadsheet for calculation of their percentages. Correction factors to convert the total amount of fatty acids in a lipid class could then be applied. Total percentages of each component were compared to the derivatized original extract prior to TLC.

Extraction Method: Dried and mechanically disrupted yeast cells (“microbial biomass”) were generally charged to an extraction vessel packed between plugs of glass wool, flushed with CO₂, and then heated and pressurized to the desired operating conditions under CO₂ flow. The CO₂ was fed directly from a commercial cylinder equipped with an eductor tube and was metered with a high-pressure pump. Pressure was maintained on the extraction vessel through use of a restrictor on the effluent side of the vessel, and the microbial oil sample was collected in a sample vessel while simultaneously venting the CO₂ solvent to the atmosphere. A cosolvent (e.g., ethanol) could optionally be added to the extraction solvent fed to the extraction vessel through use of a cosolvent pump (Isco Model 100D syringe pump). Reported extraction yields from the microbial biomass were determined gravimetrically by measuring the residual biomass and determining the total mass loss during the extraction.

Example 5 was conducted using a commercially-available automated SCF extraction instrument (Isco Model SFX3560). This instrument utilized 10-mL plastic extraction vessels equipped with a 2-micron sintered metal filter on each end of the extraction vessel. This vessel was charged with the substrate to be extracted and then loaded into a high pressure extraction chamber which equalized the pressure on the inside and outside of the extraction vessel. The CO₂ solvent was metered with a syringe pump (ISCO Model 260D), preheated to the specified extraction temperature, and then passed through the extraction vessel. The extraction chamber was heated with electrical resistance heaters to the desired extraction temperature. Pressure was maintained on the vessel with an automated variable restrictor, which was an integral part of the instrument.

Examples 1-4 and 6-9 were conducted in a custom high-pressure extraction apparatus. Extraction vessels were fabricated from 316 SS tubing and equipped with a 2-micron sintered metal filter on the effluent end of the vessel. The CO₂ was metered with a positive displacement pump equipped with a refrigerated head assembly (Jasco Model PU-1580-CO2). The extraction vessel was installed inside of a custom machined aluminum block equipped with four calrod heating cartridges which were controlled by an automated temperature controller. Extraction pressure was maintained with an automated back pressure regulator (Jasco Model BP-1580-81).

Analyses of the various lipid components in the microbial biomass, residual biomass and extracted oils, as reported in the Examples, were determined using the thin layer and gas chromatographic methods described herein above. This summary reflects analysis of the lipids extracted from the microbial biomass using the analytical procedure; however, the amount of lipids analyzed by this procedure for the residual biomass samples is relatively small when compared to that of the microbial biomass and extracted oil samples (typically <3% of the extracted oils in the initial microbial biomass).

Results are reported in summary tables showing the relative distribution of lipid components for microbial biomass, residual biomass and extracted oil samples. For each identified fatty acid shown in the horizontal row across the top of the table, the relative distribution of that component as phospholipids (PL), diacylglycerides (DAG), free fatty acids (FFA), and triacylglycerides (TAG) is shown vertically down the table columns. The first row for each sample shows analysis of the derivatized original extract prior to TLC, while the subsequent rows give the analyses of each component by TLC and GC, with the total percentages of each component presented in the far right column for that sample.

The reported extraction yield of microbial oil was determined by the weight difference between the microbial biomass before extraction and the residual biomass after extraction, expressed as a percentage. The weight difference was assumed to be due to the amount of microbial oil extracted by processing with CO₂. The actual weight of the oil obtained was generally found to be within about 85% of the weight expected based on the mass difference.

### Example 1

### Extraction Curve at 311 Bar and 40° C.

The purpose of this Example was to demonstrate generation of an extraction curve. An 8-mL extraction vessel fabricated from 316 SS tubing (0.95 cm o.d.×0.62 cm i.d.×26.7 cm long) was repeatedly charged with nominally 2.7 g of dried and mechanically disrupted yeast cells of Yarrowia lipolytica strain Y8672 (i.e., microbial biomass) for a series of extractions to determine the extraction curve for this microbial biomass at 40° C. and 311 bar. For each extraction, the extraction vessel and microbial biomass were flushed with CO₂ and then pressurized to 311 bar with CO₂ at 40° C. The microbial biomass was extracted at these conditions and a CO₂ flow rate of 1.5 g/min for various times to give a range of solvent-to-feed ratios resulting in a corresponding extraction yield, as shown in Table 4.

TABLE 4
Solvent To Feed Ratio And Extraction
Yield Data At 311 Bar And 40° C.

| Specific Solvent | Extraction |
|---|---|
| Ratio | Yield |
| (g CO₂/g Yeast) | (wt %) |
| 6.0 | 5.5 |
| 6.0 | 6.2 |
| 6.0 | 4.7 |
| 10.9 | 10.3 |
| 13.6 | 9.3 |
| 14.8 | 10.9 |
| 19.5 | 13.0 |
| 19.7 | 10.6 |
| 19.7 | 15.5 |
| 24.8 | 14.3 |
| 25.1 | 17.5 |
| 25.7 | 16.8 |
| 29.9 | 18.0 |
| 39.5 | 18.7 |
| 49.5 | 18.7 |
| 54.5 | 18.8 |
| 59.8 | 18.9 |
| 80.5 | 19.0 |
| 98.5 | 18.7 |
| 109.3 | 18.7 |
| 149.8 | 19.2 |

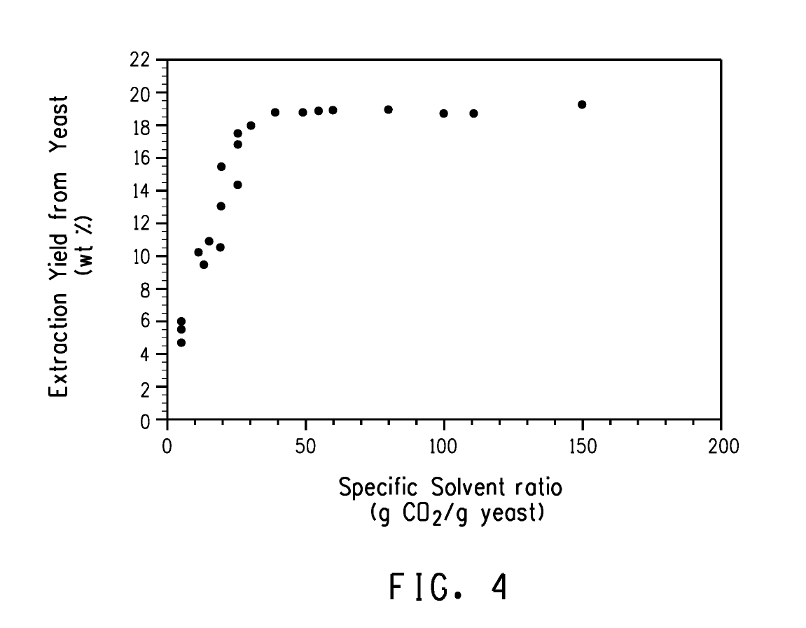

FIG. 4 plots these data in an extraction curve. The break in the curve at a solvent-to-feed ratio of about 40 g CO₂/g yeast indicates that at least this solvent ratio is required to effectively extract the available microbial oil in this particular microbial biomass at the selected temperature and pressure.

The series of extractions can be repeated at different temperature and/or pressure conditions to generate a series of extraction curves for a particular microbial biomass sample, enabling selection of the optimum extraction conditions based on economics, desired extraction yield, or total amount of CO₂ used, for example.

### Comparative Example

### Extraction of Yeast Cells without Fractionation of the Extracted Oil

The purpose of this Comparative Example was to demonstrate extraction of microbial biomass with CO₂, without fractionation of the extract or sequential extraction of the residual biomass, and to provide the lipid composition of the extract obtained.

An 18-mL extraction vessel fabricated from 316 SS tubing (1.27 cm o.d.×0.94 cm i.d.×26.0 cm long) was charged with 4.99 g of dried and mechanically disrupted yeast cells of Yarrowia lipolytica strain Y8672 (i.e., microbial biomass). The microbial biomass was flushed with CO₂, then heated to 40° C. and pressurized to 222 bar. The microbial biomass was extracted at these conditions at a flow rate of 2.3 g/min CO₂ for 5.5 hr, giving a final solvent-to-feed ratio of 149 g CO₂/g yeast. The yield of the extract was 18.2 wt %.

The Table below summarizes lipid analyses for the microbial biomass, the residual biomass, and the extracted oil.

TABLE 5
Comparative Example: Weight Percent Distribution Of Lipid Components

| | | | | | 18:3 | | 20:3 | | 20:4 | | | |
|---|---|---|---|---|---|---|---|---|---|---|---|---|
| | 16:0 | 18:0 | 18:1 | 18:2 | (n-3) | 20:2 | (n-6) | 20:4 | (n-3) | 20:5 | | |
| Sample | Palmitic | Stearic | Oleic | Linoleic | ALA | EDA | HGLA | ARA | ETA | EPA | other | Total |
| Microbial | 3 | 2 | 4 | 15 | 1 | 2 | 2 | 1 | 2 | 55 | 11 | 100 |
| Biomass | | | | | | | | | | | | |
| PL | 1 | 0 | 0 | 1 | 0 | 0 | 0 | 0 | 0 | 2 | 1 | 6 |
| DAG | 1 | 0 | 0 | 1 | 0 | 0 | 0 | 0 | 0 | 2 | 1 | 6 |
| FFA | 0 | 0 | 0 | 0 | 0 | 0 | 0 | 0 | 0 | 3 | 1 | 6 |
| TAG | 1 | 2 | 4 | 13 | 1 | 1 | 1 | 1 | 1 | 49 | 7 | 82 |
| Sum | 3 | 2 | 5 | 16 | 1 | 2 | 2 | 1 | 1 | 56 | 9 | 100 |
| Residual | 7 | 4 | 4 | 18 | 1 | 2 | 2 | 1 | 1 | 49 | 10 | 100 |
| Biomass | | | | | | | | | | | | |
| PL | 6 | 3 | 2 | 10 | 1 | 1 | 1 | 0 | 1 | 14 | 5 | 43 |
| DAG | 0 | 0 | 0 | 1 | 0 | 0 | 0 | 0 | 0 | 2 | 1 | 4 |
| FFA | 1 | 0 | 0 | 1 | 0 | 0 | 0 | 0 | 0 | 6 | 1 | 12 |
| TAG | 1 | 1 | 2 | 7 | 1 | 1 | 1 | 1 | 0 | 23 | 4 | 41 |
| Sum | 8 | 4 | 4 | 18 | 1 | 2 | 2 | 1 | 2 | 45 | 11 | 100 |
| Extracted Oil | 2 | 2 | 4 | 14 | 1 | 2 | 3 | 3 | 1 | 56 | 10 | 100 |
| PL | 0 | 0 | 0 | 0 | 0 | 0 | 0 | 0 | 0 | 0 | 0 | 0 |
| DAG | 1 | 0 | 0 | 1 | 0 | 0 | 0 | 0 | 0 | 2 | 1 | 6 |
| FFA | 0 | 0 | 0 | 0 | 0 | 0 | 0 | 0 | 0 | 2 | 1 | 4 |
| TAG | 1 | 2 | 4 | 14 | 1 | 2 | 2 | 1 | 1 | 53 | 8 | 90 |
| Sum | 2 | 2 | 5 | 16 | 1 | 2 | 2 | 1 | 1 | 58 | 9 | 100 |

For the yeast microbial biomass, 50 weight percent [“wt %”] of the FFAs and 59.8 wt % of the TAGs were found to contain EPA. Specifically, the wt % EPA within FFAs was calculated as the wt % of FFAs comprising EPA in the microbial biomass (i.e., 3) divided by the total wt % of FFAs in the microbial biomass (i.e., 6), expressed as a percentage and with both percent values taken from the TLC analysis, as shown above in Table 5. Similarly, the wt % EPA within TAGs was calculated as the wt % of TAGs comprising EPA in the microbial biomass (i.e., 49) divided by the total wt % of TAGs in the microbial biomass (i.e., 82), expressed as a percentage and with both percent values taken from the TLC analysis, supra.

The absence (i.e., 0 wt %) of PLs in the extracted oil shows that the PL fraction of the lipids present in the initial microbial biomass remains in the residual biomass (43 wt % PLs) and does not partition with the CO₂ into the extracted oil. Additionally, the extracted oil is enriched in TAGs (i.e., 90 wt %) when compared to the TAG content of the microbial biomass (i.e., 82 wt %). Thus, the extracted oil is a refined lipid composition.

More specifically, the results show the refined lipid composition of the extracted oil contains 90 wt % TAGs, 4 wt % FFAs, and 6 wt % DAGs, wherein 50% of the FFAs and 58.9% of the TAGs were found to contain EPA. More specifically, the wt % EPA within FFAs was calculated as the wt % of FFAs comprising EPA in the extracted oil (i.e., 2) divided by the total wt % of FFAs in the extracted oil (i.e., 4); and, the wt % EPA within TAGs was calculated as the wt % of TAGs comprising EPA in the extracted oil (i.e., 53) divided by the total wt % of TAGs in the extracted oil (i.e., 90). The refined lipid composition is not enriched in EPA relative to the microbial biomass.

### Examples 2-4

### Lipid Fractionation by Sequential Pressure Extraction

The purpose of Examples 2, 3 and 4 was to demonstrate sequential pressure extraction of microbial biomass under various extraction conditions and to provide the lipid compositions of the extracted oils obtained.

Examples 2, 3 and 4 collectively illustrate that partitioning of the lipid components of the extracted oil can be influenced by the selection of the extraction conditions in a multi-step extraction. Such partitioning would likewise result from a sequential reduction of pressure of the extracted oil obtained by a process as illustrated in FIG. 3.

These results obtained in Examples 2, 3 and 4 are expected to be similar to the results which could be obtained by SCF CO₂-extraction of the microbial biomass, wherein the extracted oil is subsequently fractionated via stepwise pressure reduction.

### Example 2

### 125 Bar to 222 Bar

An 18-mL extraction vessel fabricated from 316 SS tubing (1.27 cm o.d.×0.94 cm i.d.×26.0 cm long) was charged with 3.50 g of dried and mechanically disrupted yeast cells of Yarrowia lipolytica strain Y8672 as the microbial biomass.

Extract A: The microbial biomass was flushed with CO₂, then heated to 40° C. and pressurized to 125 bar. The microbial biomass was extracted at these conditions at a flow rate of 2.3 g/min CO₂ for 5 hrs, at which time the pressure was increased to 150 bar. The extraction was continued for an additional 1.2 hrs, giving a final solvent-to-feed ratio of 238 g CO₂/g yeast. The yield of Extract A was 11.7 wt %.

Extract B: The extraction was continued with the same partially extracted microbial biomass by increasing the pressure to 222 bar and continuing the CO₂ flow at 2.3 g/min for 4.0 hrs, giving a final solvent-to-feed ratio of 153 g CO₂/g yeast for this fraction. The yield of Extract B was 6.2 wt % of the original microbial biomass charged to the extraction vessel.

The Table below summarizes lipid analyses for the microbial biomass and the two extracted oil fractions (i.e., Extract A and Extract B).

TABLE 6
Example 2: Weight Percent Distribution of Lipid Components

| | | | | | 18:3 | | 20:3 | | 20:4 | | | |
|---|---|---|---|---|---|---|---|---|---|---|---|---|
| | 16:0 | 18:0 | 18:1 | 18:2 | (n-3) | 20:2 | (n-6) | 20:4 | (n-3) | 20:5 | | |
| Sample | palmitic | Stearic | Oleic | Linoleic | ALA | EDA | HGLA | ARA | ETA | EPA | other | Total % |
| Microbial | 3 | 2 | 4 | 15 | 1 | 2 | 2 | 1 | 2 | 55 | 11 | 100 |
| Biomass | | | | | | | | | | | | |
| PL | 1 | 0 | 0 | 1 | 0 | 0 | 0 | 0 | 0 | 2 | 1 | 6 |
| DAG | 1 | 0 | 0 | 1 | 0 | 0 | 0 | 0 | 0 | 2 | 1 | 6 |
| FFA | 0 | 0 | 0 | 0 | 0 | 0 | 0 | 0 | 0 | 3 | 1 | 6 |
| TAG | 1 | 2 | 4 | 13 | 1 | 1 | 1 | 1 | 1 | 49 | 7 | 82 |
| Sum | 3 | 2 | 5 | 16 | 1 | 2 | 2 | 1 | 1 | 56 | 9 | 100 |
| Extract A | 3 | 2 | 5 | 16 | 1 | 2 | 2 | 1 | 1 | 56 | 10 | |
| PL | 0 | 0 | 0 | 0 | 0 | 0 | 0 | 0 | 0 | 0 | 0 | 0 |
| DAG | 1 | 0 | 0 | 2 | 0 | 0 | 0 | 0 | 0 | 3 | 1 | 9 |
| FFA | 1 | 0 | 0 | 1 | 0 | 0 | 0 | 0 | 0 | 4 | 1 | 9 |
| TAG | 2 | 2 | 5 | 15 | 1 | 1 | 1 | 1 | 1 | 44 | 7 | 82 |
| Sum | 4 | 3 | 6 | 18 | 1 | 2 | 2 | 1 | 1 | 51 | 10 | 100 |
| Extract B | 1 | 2 | 4 | 13 | 1 | 2 | 2 | 1 | 2 | 62 | 9 | |
| PL | 0 | 0 | 0 | 0 | 0 | 0 | 0 | 0 | 0 | 0 | 0 | 0 |
| DAG | 0 | 0 | 0 | 0 | 0 | 0 | 0 | 0 | 0 | 0 | 0 | 1 |
| FFA | 0 | 0 | 0 | 0 | 0 | 0 | 0 | 0 | 0 | 0 | 0 | 0 |
| TAG | 1 | 2 | 4 | 14 | 1 | 2 | 2 | 1 | 1 | 62 | 8 | 99 |
| Sum | 1 | 2 | 5 | 14 | 1 | 2 | 2 | 1 | 1 | 62 | 8 | 100 |

Under the extraction conditions employed, some TAGs (i.e., 82 wt %) and most of the FFAs and DAGs of the extracted oil selectively partitioned into Extract A (which contains 9 wt % of each). The PL fraction of the lipids present in the initial microbial biomass did not partition with the CO₂ into Extract A. Thus, Extract A is a refined lipid composition.

Under the extraction conditions employed, Extract B was enriched in TAGs (and contains only 1 wt % DAGs and no measured FFAs) and was substantially free of PLs. Thus, the extracted oil of Extract B is a refined lipid composition enriched in TAGs. More specifically, Extract B comprised 99% TAGs, of which 62.6% contained EPA (i.e., calculated as the wt % of TAGs comprising EPA in Extract B [i.e., 62] divided by the total wt % of TAGs in Extract B [i.e., 99], expressed as a percentage, and with both percent values taken from the TLC analysis). In contrast, EPA was present in about 59.8% of the TAGs in the microbial biomass (i.e., calculated as the wt % of TAGs comprising EPA in the microbial biomass [i.e., 49] divided by the total wt % of TAGs in the microbial biomass [i.e., 82]). The refined lipid composition of Extract B is therefore enriched in EPA, a C20 PUFA, relative to the microbial biomass.

### Example 3

### 125 Bar to 141 Bar to 222 Bar

An 89-mL extraction vessel fabricated from 316 SS tubing (2.54 cm o.d.×1.93 cm i.d.×30.5 cm long) was charged with 15.0 g of dried and mechanically disrupted yeast cells of Yarrowia lipolytica strain Y8672 as the microbial biomass.

Extract A: The microbial biomass was flushed with CO₂, then heated to 40° C. and pressurized to 125 bar. The microbial biomass was extracted at these conditions at a flow rate of 2.3 g/min CO₂ for 3.9 hrs, at which time the flow rate was increased to 4.7 g/min CO₂ and the extraction was continued for an additional 2.3 hrs. The pressure was then increased to 141 bar. The extraction was continued for an additional 4.1 hrs at 4.7 g/min CO₂, giving a final solvent-to-feed ratio of 154 g CO₂/g yeast. The yield of Extract A was 8.7 wt %.

Extract B: The extraction was continued with the same partially extracted microbial biomass by increasing the pressure to 222 bar and continuing the CO₂ flow at 4.7 g/min for 8.0 hrs, giving a final solvent-to-feed ratio of 150 g CO₂/g yeast for this extract. The yield of Extract B was 15.4 wt % of the original microbial biomass charged to the extraction vessel.

The Table below summarizes lipid analyses for the microbial biomass, the residual biomass after the final extraction, and the two extracted oil fractions (i.e., Extract A and Extract B).

TABLE 7
Example 3: Weight Percent Distribution of Lipid Components

| | | | | | 18:3 | | 20:3 | | 20:4 | | | |
|---|---|---|---|---|---|---|---|---|---|---|---|---|
| | 16:0 | 18:0 | 18:1 | 18:2 | (n-3) | 20:2 | (n-6) | 20:4 | (n-3) | 20:5 | | |
| Sample | palmitic | Stearic | Oleic | Linoleic | ALA | EDA | HGLA | ARA | ETA | EPA | other | Total |
| Microbial | 3 | 2 | 4 | 15 | 1 | 2 | 2 | 1 | 2 | 55 | 11 | 100 |
| Biomass | | | | | | | | | | | | |
| PL | 1 | 0 | 0 | 1 | 0 | 0 | 0 | 0 | 0 | 2 | 1 | 6 |
| DAG | 1 | 0 | 0 | 1 | 0 | 0 | 0 | 0 | 0 | 2 | 1 | 6 |
| FFA | 0 | 0 | 0 | 0 | 0 | 0 | 0 | 0 | 0 | 3 | 1 | 6 |
| TAG | 1 | 2 | 4 | 13 | 1 | 1 | 1 | 1 | 1 | 49 | 7 | 82 |
| Sum | 3 | 2 | 5 | 16 | 1 | 2 | 2 | 1 | 1 | 56 | 9 | 100 |
| Residual | 6 | 3 | 4 | 15 | 1 | 3 | 2 | 1 | 3 | 43 | 15 | 100 |
| Biomass | | | | | | | | | | | | |
| PL | 5 | 2 | 1 | 9 | 0 | 0 | 1 | 0 | 1 | 12 | 5 | 37 |
| DAG | 0 | 0 | 0 | 1 | 0 | 0 | 0 | 0 | 0 | 2 | 1 | 5 |
| FFA | 1 | 1 | 0 | 1 | 0 | 1 | 0 | 0 | 0 | 8 | 2 | 15 |
| TAG | 1 | 1 | 2 | 7 | 1 | 1 | 1 | 0 | 1 | 24 | 4 | 42 |
| Sum | 7 | 4 | 4 | 17 | 1 | 2 | 2 | 1 | 2 | 46 | 11 | 100 |
| Extract A | 3 | 2 | 5 | 16 | 1 | 2 | 2 | 1 | 2 | 56 | 9 | 100 |
| PL | 0 | 0 | 0 | 0 | 0 | 0 | 0 | 0 | 0 | 0 | 0 | 0 |
| DAG | 1 | 1 | 0 | 2 | 0 | 0 | 0 | 0 | 0 | 4 | 1 | 11 |
| FFA | 1 | 0 | 0 | 1 | 0 | 0 | 0 | 0 | 0 | 5 | 1 | 9 |
| TAG | 2 | 2 | 4 | 14 | 1 | 1 | 1 | 1 | 1 | 45 | 7 | 80 |
| Sum | 3 | 3 | 5 | 17 | 1 | 2 | 2 | 1 | 1 | 55 | 9 | 100 |
| Extract B | 1 | 2 | 5 | 14 | 1 | 2 | 2 | 1 | 1 | 61 | 9 | 100 |
| PL | 0 | 0 | 0 | 0 | 0 | 0 | 0 | 0 | 0 | 0 | 0 | 1 |
| DAG | 0 | 0 | 0 | 0 | 0 | 0 | 0 | 0 | 0 | 1 | 0 | 2 |
| FFA | 0 | 0 | 0 | 0 | 0 | 0 | 0 | 0 | 0 | 0 | 0 | 0 |
| TAG | 1 | 2 | 5 | 15 | 1 | 2 | 2 | 1 | 1 | 60 | 8 | 97 |
| Sum | 1 | 2 | 5 | 15 | 1 | 2 | 2 | 1 | 1 | 61 | 8 | 100 |

The results show that the PL fraction of the lipids present in the microbial biomass remains in the residual biomass (i.e., 37 wt % PLs in the residual biomass versus 0 wt % in Extract A and 1 wt % in Extract B).

Under the extraction conditions employed, the FFAs and DAGs of the microbial biomass selectively partitioned into the refined lipid composition of Extract A, while the refined lipid composition Extract B was enriched in TAGs. More specifically, Extract B was about 97% TAGs with no measured FFAs, and about 61.9% of the TAGs were found to contain EPA. In contrast, EPA was present in about 59.8% of the TAGs in the microbial biomass (i.e., calculated as the wt % of TAGs comprising EPA in the microbial biomass [i.e., 49] divided by the total wt % of TAGs in the microbial biomass [i.e., 82]). Thus, the refined lipid composition of Extract B is therefore enriched in EPA, a C20 PUFA, relative to the microbial biomass.

### Example 4

### 110 Bar to 222 Bar

An 89-mL extraction vessel fabricated from 316 SS tubing (2.54 cm o.d.×1.93 cm i.d.×30.5 cm long) was charged with 20.0 g of dried and mechanically disrupted yeast cells of Yarrowia lipolytica strain Y8672 as the microbial biomass.

Extract A: The microbial biomass was flushed with CO₂, then heated to 40° C. and pressurized to 110 bar. The microbial biomass was extracted at these conditions at a flow rate of 4.7 g/min CO₂ for 7.1 hrs, giving a final solvent-to-feed ratio of 100 g CO₂/g yeast. The yield of Extract A was 4.1 wt %.

Extract B: The extraction was continued with the same partially extracted microbial biomass by increasing the pressure to 222 bar and continuing the CO₂ flow at 4.7 g/min for 15.0 hrs, giving a final solvent-to-feed ratio of 212 g CO₂/g yeast for this extract. The yield of Extract B was 14.6 wt % of the original microbial biomass charged to the extraction vessel.

The Table below summarizes lipid analyses for the starting feed yeast, the residual biomass after the final extraction, and the two extracted oil fractions (i.e., Extract A and Extract B).

TABLE 8
Example 4: Weight Percent Distribution of Lipid Components

| | | | | | 18:3 | | 20:3 | | 20:4 | | | |
|---|---|---|---|---|---|---|---|---|---|---|---|---|
| | 16:0 | 18:0 | 18:1 | 18:2 | (n-3) | 20:2 | (n-6) | 20:4 | (n-3) | 20:5 | | |
| Sample | palmitic | Stearic | Oleic | Linoleic | ALA | EDA | HGLA | ARA | ETA | EPA | other | Total |
| Microbial | 3 | 2 | 4 | 15 | 1 | 2 | 2 | 1 | 2 | 55 | 11 | 100 |
| Biomass | | | | | | | | | | | | |
| PL | 1 | 0 | 0 | 1 | 0 | 0 | 0 | 0 | 0 | 2 | 1 | 6 |
| DAG | 1 | 0 | 0 | 1 | 0 | 0 | 0 | 0 | 0 | 2 | 1 | 6 |
| FFA | 0 | 0 | 0 | 0 | 0 | 0 | 0 | 0 | 0 | 3 | 1 | 6 |
| TAG | 1 | 2 | 4 | 13 | 1 | 1 | 1 | 1 | 1 | 49 | 7 | 82 |
| Sum | 3 | 2 | 5 | 16 | 1 | 2 | 2 | 1 | 1 | 56 | 9 | 100 |
| Residual | 6 | 3 | 4 | 15 | 1 | 2 | 3 | 5 | 2 | 43 | 13 | 100 |
| Biomass | | | | | | | | | | | | |
| PL | 5 | 2 | 2 | 9 | 1 | 1 | 1 | 0 | 1 | 13 | 5 | 41 |
| DAG | 0 | 0 | 0 | 1 | 0 | 0 | 0 | 0 | 0 | 2 | 1 | 4 |
| FFA | 1 | 0 | 0 | 1 | 0 | 0 | 0 | 0 | 0 | 6 | 1 | 12 |
| TAG | 1 | 1 | 2 | 7 | 1 | 1 | 1 | 1 | 1 | 25 | 4 | 43 |
| Sum | 7 | 4 | 4 | 18 | 1 | 2 | 2 | 1 | 2 | 46 | 11 | 100 |
| Extract A | 5 | 3 | 4 | 15 | 1 | 2 | 2 | 1 | 1 | 56 | 8 | 100 |
| PL | 0 | 0 | 0 | 0 | 0 | 0 | 0 | 0 | 0 | 0 | 0 | 0 |
| DAG | 2 | 1 | 1 | 3 | 0 | 0 | 0 | 0 | 0 | 5 | 2 | 13 |
| FFA | 2 | 1 | 1 | 3 | 0 | 1 | 1 | 1 | 1 | 22 | 3 | 37 |
| TAG | 1 | 1 | 3 | 9 | 1 | 1 | 1 | 0 | 1 | 27 | 4 | 49 |
| Sum | 6 | 3 | 5 | 15 | 1 | 2 | 2 | 1 | 1 | 53 | 9 | 100 |
| Extract B | 2 | 2 | 5 | 15 | 1 | 2 | 2 | 1 | 1 | 60 | 9 | 100 |
| PL | 0 | 0 | 0 | 0 | 0 | 0 | 0 | 0 | 0 | 0 | 0 | 0 |
| DAG | 0 | 0 | 0 | 1 | 0 | 0 | 0 | 0 | 0 | 2 | 0 | 4 |
| FFA | 0 | 0 | 0 | 0 | 0 | 0 | 0 | 0 | 0 | 0 | 0 | 1 |
| TAG | 1 | 2 | 5 | 16 | 1 | 2 | 2 | 1 | 1 | 56 | 8 | 95 |
| Sum | 2 | 2 | 5 | 16 | 1 | 2 | 2 | 1 | 1 | 58 | 8 | 100 |

The results show that the PL fraction of the lipids present in the microbial biomass remains in the residual biomass (i.e., 41 wt % PLs in the residual biomass versus 0 wt % in Extract A and Extract B).

Under the extraction conditions employed, the FFAs and DAGs of the microbial biomass selectively partitioned into the refined lipid composition of Extract A, while the refined lipid composition of Extract B was enriched in TAGs. More specifically, Extract B was about 95% TAGs with no measured FFAs, and about 58.9% of the TAGs were found to contain EPA. The refined lipid composition of Extract B is not enriched in EPA relative to the microbial biomass.

### Examples 5-7

### Supercritical Fluid CO₂ Extraction at Various Pressures

The purpose of Examples 5, 6 and 7 was to demonstrate extraction of microbial biomass with CO₂ as a supercritical fluid (SCF) at various pressures (i.e., 500 bar, 310 bar and 222 bar, respectively), and provide the lipid composition of the extracted oils obtained. Such extraction conditions could be used in the first step of a method for obtaining a refined composition comprising at least one PUFA, where the method comprises processing microbial biomass comprising at least one PUFA with CO₂ under suitable extraction conditions, and subsequently fractionating the extract, for example by sequential pressure reduction.

### Example 5

### SCF CO₂ at 500 Bar

A 10-mL extraction vessel was charged with 2.01 g of dried and mechanically disrupted yeast cells of Yarrowia lipolytica strain Y4305-F1B1 as the microbial biomass, and the vessel was mounted in an Isco Model SFX3560 extractor. The microbial biomass was flushed with CO₂, then heated to 40° C. and pressurized to 500 bar. The microbial biomass was extracted at these conditions at a flow rate of 0.86 g/min CO₂ for 5.8 hrs, giving a final solvent-to-feed ratio of 150 g CO₂/g yeast. The yield of extracted oil was 32.8 wt %. The Table below summarizes lipid analyses for the microbial biomass, the residual biomass, and the extracted oil.

TABLE 9
Example 5: Weight Percent Distribution of Lipid Components

| | | | | | 18:3 | | 20:3 | | 20:4 | | | |
|---|---|---|---|---|---|---|---|---|---|---|---|---|
| | 16:0 | 18:0 | 18:1 | 18:2 | (n-3) | 20:2 | (n-6) | 20:4 | (n-3) | 20:5 | | |
| Sample | palmitic | Stearic | Oleic | Linoleic | ALA | EDA | HGLA | ARA | ETA | EPA | other | Total |
| Microbial | 3 | 3 | 6 | 21 | 4 | 4 | 2 | 1 | 2 | 44 | 9 | 100 |
| Biomass | | | | | | | | | | | | |
| PL | 1 | 0 | 0 | 1 | 0 | 0 | 0 | 0 | 0 | 1 | 0 | 4 |
| DAG | 0 | 0 | 0 | 2 | 0 | 0 | 0 | 0 | 0 | 2 | 1 | 6 |
| FFA | 0 | 0 | 0 | 1 | 0 | 0 | 0 | 0 | 0 | 2 | 1 | 6 |
| TAG | 2 | 2 | 5 | 18 | 3 | 3 | 2 | 0 | 1 | 38 | 7 | 84 |
| Sum | 3 | 3 | 6 | 22 | 4 | 3 | 2 | 1 | 2 | 43 | 9 | 100 |
| Residual | 9 | 4 | 5 | 24 | 3 | 3 | 2 | 1 | 2 | 36 | 7 | 100 |
| Biomass | | | | | | | | | | | | |
| PL | 7 | 3 | 2 | 14 | 1 | 1 | 1 | 0 | 1 | 11 | 3 | 48 |
| DAG | 0 | 0 | 0 | 1 | 0 | 0 | 0 | 0 | 0 | 1 | 0 | 4 |
| FFA | 1 | 1 | 0 | 2 | 0 | 1 | 0 | 0 | 0 | 4 | 2 | 12 |
| TAG | 1 | 1 | 2 | 8 | 1 | 1 | 1 | 0 | 1 | 15 | 3 | 36 |
| Sum | 9 | 5 | 5 | 25 | 3 | 3 | 2 | 1 | 2 | 32 | 9 | 100 |
| Extracted Oil | 2 | 2 | 6 | 21 | 4 | 4 | 2 | 1 | 2 | 46 | 9 | 100 |
| PL | 0 | 0 | 0 | 0 | 0 | 0 | 0 | 0 | 0 | 0 | 0 | 0 |
| DAG | 0 | 0 | 0 | 2 | 0 | 0 | 0 | 0 | 0 | 2 | 1 | 7 |
| FFA | 0 | 0 | 0 | 1 | 0 | 0 | 0 | 0 | 0 | 2 | 1 | 5 |
| TAG | 2 | 2 | 5 | 19 | 3 | 3 | 2 | 0 | 1 | 40 | 7 | 88 |
| Sum | 3 | 3 | 6 | 21 | 4 | 4 | 2 | 1 | 2 | 45 | 9 | 100 |

The results of Table 9 show that the PL fraction of the lipids present in the microbial biomass remained in the residual biomass and did not partition into the CO₂-extracted oil (i.e., 48 wt % PLs in the residual biomass versus 0 wt % in the extracted oil). Since the extracted oil was also enriched in TAGs relative to the microbial biomass, this oil was a refined lipid composition. The refined lipid composition comprised 5 wt % FFAs, 7 wt % DAGs, and 88 wt % TAGs.

### Example 6

### SCF CO₂ Extraction at 310 Bar

An 89-mL extraction vessel fabricated from 316 SS tubing (2.54 cm o.d.×1.93 cm i.d.×30.5 cm long) was charged with 25.1 g of dried and mechanically disrupted yeast cells of Yarrowia lipolytica strain Y9502 as the microbial biomass. The microbial biomass was flushed with CO₂, then heated to 40° C. and pressurized to 310 bar. The microbial biomass was extracted at these conditions at a flow rate of 5.0 mL/min CO₂ for 4.4 hrs, giving a final solvent-to-feed ratio of 50 g CO₂/g yeast. The yield of extracted oil was 28.8 wt %. The Table below summarizes lipid analyses for the microbial biomass, the residual biomass, and the extracted oil.

TABLE 10
Example 6: Weight Percent Distribution of Lipid Components

| | | | | | 18:3 | | 20:3 | | | | | |
|---|---|---|---|---|---|---|---|---|---|---|---|---|
| | 16:0 | 16:1 | 18:1 | 18:2 | (n-3) | 20:2 | (n-6) | 20:4 | | 20:5 | | |
| Sample | palmitic | Palmitoleic | Oleic | Linoleic | ALA | EDA | HGLA | ARA | EtrA | EPA | other | Total |
| Microbial | 2 | 1 | 4 | 10 | 1 | 5 | 6 | 2 | 0 | 51 | 12 | 100 |
| Biomass | | | | | | | | | | | | |
| PL | 0 | 0 | 0 | 1 | 0 | 0 | 0 | 0 | 0 | 2 | 1 | 7 |
| DAG | 0 | 0 | 0 | 1 | 0 | 0 | 0 | 0 | 0 | 3 | 1 | 7 |
| FFA | 1 | 0 | 0 | 1 | 0 | 2 | 1 | 0 | 0 | 8 | 1 | 15 |
| TAG | 1 | 0 | 3 | 9 | 0 | 2 | 4 | 1 | 0 | 41 | 6 | 72 |
| Sum | 2 | 1 | 4 | 11 | 1 | 5 | 6 | 1 | 1 | 53 | 9 | 100 |
| Residual | 4 | 1 | 4 | 12 | 0 | 5 | 6 | 2 | 3 | 48 | 10 | 100 |
| Biomass | | | | | | | | | | | | |
| PL | 3 | 0 | 2 | 6 | 0 | 2 | 2 | 0 | 1 | 14 | 5 | 40 |
| DAG | 0 | 0 | 1 | 1 | 0 | 1 | 1 | 0 | 0 | 3 | 1 | 9 |
| FFA | 0 | 0 | 0 | 0 | 0 | 2 | 1 | 0 | 0 | 6 | 2 | 14 |
| TAG | 0 | 0 | 2 | 4 | 0 | 2 | 3 | 1 | 0 | 20 | 4 | 38 |
| Sum | 4 | 1 | 5 | 12 | 1 | 6 | 6 | 2 | 2 | 44 | 12 | 100 |
| Extracted Oil | 2 | 1 | 4 | 11 | 1 | 5 | 6 | 1 | 0 | 56 | 9 | 100 |
| PL | 0 | 0 | 0 | 0 | 0 | 0 | 0 | 0 | 0 | 0 | 0 | 1 |
| DAG | 1 | 0 | 0 | 1 | 0 | 0 | 0 | 0 | 0 | 2 | 1 | 6 |
| FFA | 1 | 0 | 0 | 1 | 0 | 2 | 1 | 0 | 0 | 8 | 2 | 16 |
| TAG | 1 | 1 | 4 | 10 | 1 | 2 | 4 | 1 | 0 | 43 | 7 | 77 |
| Sum | 2 | 1 | 4 | 12 | 1 | 5 | 6 | 1 | 0 | 53 | 9 | 100 |

The results of Table 10 show that most of the PL fraction of the lipids present in the microbial biomass remained in the residual biomass and did not partition into the CO₂-extracted oil (i.e., 40 wt % PLs in the residual biomass versus 1 wt % in the extracted oil). Since the extracted oil was substantially free of PLs and enriched in TAGs relative to the microbial biomass, this oil was a refined lipid composition. The refined lipid composition comprised 16 wt % FFAs, 6 wt % DAGs, and 77 wt % TAGs.

### Example 7

### SCF CO₂ Extraction at 222 Bar

An 89-mL extraction vessel fabricated from 316 SS tubing (2.54 cm o.d.×1.93 cm i.d.×30.5 cm long) was charged with 25.1 g of dried and mechanically disrupted yeast cells of Yarrowia lipolytica strain Y8672 as the microbial biomass. The microbial biomass was flushed with CO₂, then heated to 40° C. and pressurized to 222 bar. The microbial biomass was extracted at these conditions at a flow rate of 4.7 g/min CO₂ for 13.7 hrs, giving a final solvent-to-feed ratio of 154 g CO₂/g yeast. The yield of extracted oil was 18.1 wt %.

This extraction supra was replicated an additional four times, each time with a fresh sample of microbial biomass. The five residual biomass samples and the five extracted oil samples were consolidated and mixed to provide composite samples from the five extractions.

The Table below summarizes lipid analyses for the microbial biomass, the consolidated residual biomass, and the consolidated extracted oil.

TABLE 11
Example 7: Weight Percent Distribution of Lipid Components

| | | | | | 18:3 | | 20:3 | | 20:4 | | | |
|---|---|---|---|---|---|---|---|---|---|---|---|---|
| | 16:0 | 18:0 | 18:1 | 18:2 | (n-3) | 20:2 | (n-6) | 20:4 | (n-3) | 20:5 | | |
| Sample | palmitic | Stearic | Oleic | Linoleic | ALA | EDA | HGLA | ARA | ETA | EPA | other | Total |
| Microbial | 3 | 3 | 4 | 15 | 1 | 2 | 4 | 1 | 3 | 51 | 11 | 100 |
| Biomass | | | | | | | | | | | | |
| PL | 1 | 0 | 0 | 2 | 0 | 0 | 0 | 0 | 0 | 2 | 1 | 8 |
| DAG | 1 | 0 | 0 | 1 | 0 | 0 | 0 | 0 | 0 | 2 | 1 | 6 |
| FFA | 0 | 0 | 0 | 0 | 0 | 0 | 0 | 0 | 0 | 3 | 1 | 7 |
| TAG | 1 | 2 | 4 | 12 | 1 | 2 | 3 | 1 | 2 | 45 | 7 | 79 |
| Sum | 3 | 3 | 5 | 16 | 1 | 2 | 3 | 1 | 3 | 53 | 9 | 100 |
| Residual | 9 | 4 | 4 | 18 | 1 | 2 | 3 | 1 | 4 | 36 | 14 | 100 |
| Biomass | | | | | | | | | | | | |
| PL | 8 | 4 | 3 | 14 | 1 | 1 | 2 | 0 | 2 | 16 | 8 | 59 |
| DAG | 0 | 0 | 0 | 1 | 0 | 0 | 0 | 0 | 0 | 1 | 1 | 3 |
| FFA | 1 | 0 | 0 | 1 | 0 | 0 | 0 | 0 | 0 | 5 | 2 | 11 |
| TAG | 1 | 1 | 1 | 4 | 0 | 1 | 1 | 0 | 1 | 13 | 4 | 27 |
| Sum | 10 | 5 | 5 | 19 | 1 | 2 | 3 | 1 | 3 | 36 | 14 | 100 |
| Extracted Oil | 2 | 2 | 4 | 14 | 1 | 2 | 4 | 1 | 4 | 52 | 12 | 100 |
| PL | 0 | 0 | 0 | 0 | 0 | 0 | 0 | 0 | 0 | 0 | 0 | 0 |
| DAG | 1 | 0 | 0 | 1 | 0 | 0 | 0 | 0 | 0 | 3 | 1 | 7 |
| FFA | 0 | 0 | 0 | 0 | 0 | 0 | 0 | 0 | 0 | 3 | 1 | 7 |
| TAG | 2 | 2 | 4 | 13 | 1 | 2 | 3 | 1 | 2 | 48 | 7 | 86 |
| Sum | 3 | 3 | 5 | 15 | 1 | 3 | 3 | 1 | 3 | 54 | 9 | 100 |

The results of Table 11 show that the PL fraction of the lipids present in the microbial biomass remained in the residual biomass and did not partition into the CO₂-extracted oil (i.e., 59 wt % PLs in the residual biomass versus 0 wt % in the extracted oil). Since the extracted oil was also enriched in TAGs relative to the microbial biomass, this oil was a refined lipid composition. The refined lipid composition comprised 7 wt % FFAs, 7 wt % DAGs, and 86 wt % TAGs.

### Example 8

### Liquid CO₂ Extraction at 85 Bar

The purpose of this Example was to demonstrate extraction of a microbial biomass with CO₂ as a liquid at 85 bar, and to provide the composition of the extracted oil obtained. Such extraction conditions could be used in the first step of a method for obtaining a refined composition comprising at least one PUFA, where the method comprises processing microbial biomass comprising at least one PUFA with CO₂ under suitable extraction conditions, and subsequently fractionating the extract, for example by sequential pressure reduction.

An 8-mL extraction vessel fabricated from 316 SS tubing (0.95 cm o.d.×0.62 cm i.d.×26.7 cm long) was charged with 0.966 g of dried and mechanically disrupted yeast cells of Yarrowia lipolytica strain Y8672 as the microbial biomass. The microbial biomass was flushed with CO₂, and then pressurized to 85 bar with liquid CO₂ at 22° C. The microbial biomass was extracted at these conditions at a flow rate of 0.69 g/min CO₂ for 8.5 hrs, giving a final solvent-to-feed ratio of 361 g CO₂/g yeast. The yield of extracted oil was 21.4 wt %.

The Table below summarizes lipid analyses for the microbial biomass, the residual biomass, and the extracted oil.

TABLE 12
Example 8: Weight Percent Distribution of Lipid Components

| | | | | | 18:3 | | 20:3 | | 20:4 | | | |
|---|---|---|---|---|---|---|---|---|---|---|---|---|
| | 16:0 | 18:0 | 18:1 | 18:2 | (n-3) | 20:2 | (n-6) | 20:4 | (n-3) | 20:5 | | |
| Sample | palmitic | Stearic | Oleic | Linoleic | ALA | EDA | HGLA | ARA | ETA | EPA | other | Total |
| Microbial | 2 | 2 | 4 | 13 | 1 | 3 | 4 | 1 | 2 | 55 | 11 | 100 |
| Biomass | | | | | | | | | | | | |
| PL | 1 | 0 | 0 | 1 | 0 | 0 | 0 | 0 | 0 | 2 | 1 | 6 |
| DAG | 0 | 0 | 0 | 1 | 0 | 0 | 0 | 0 | 0 | 2 | 1 | 5 |
| FFA | 0 | 0 | 0 | 1 | 0 | 1 | 1 | 0 | 0 | 4 | 3 | 11 |
| TAG | 1 | 2 | 4 | 11 | 1 | 2 | 2 | 1 | 1 | 45 | 7 | 77 |
| Sum | 3 | 3 | 4 | 14 | 1 | 3 | 3 | 1 | 2 | 53 | 12 | 100 |
| Residual | 5 | 4 | 3 | 14 | 1 | 3 | 4 | 1 | 3 | 47 | 13 | 100 |
| Biomass | | | | | | | | | | | | |
| PL | 3 | 2 | 1 | 5 | 0 | 0 | 1 | 0 | 1 | 10 | 4 | 28 |
| DAG | 0 | 0 | 0 | 0 | 0 | 0 | 0 | 0 | 0 | 1 | 0 | 2 |
| FFA | 0 | 0 | 0 | 1 | 0 | 1 | 0 | 0 | 0 | 7 | 2 | 13 |
| TAG | 1 | 2 | 3 | 8 | 1 | 1 | 2 | 1 | 1 | 31 | 6 | 57 |
| Sum | 4 | 4 | 4 | 15 | 1 | 3 | 3 | 1 | 3 | 49 | 12 | 100 |
| Extracted Oil | 2 | 2 | 4 | 14 | 1 | 3 | 3 | 1 | 2 | 56 | 11 | 100 |
| PL | 0 | 0 | 0 | 0 | 0 | 0 | 0 | 0 | 0 | 0 | 0 | 0.7 |
| DAG | 0 | 0 | 0 | 1 | 0 | 0 | 0 | 0 | 0 | 2 | 1 | 5 |
| FFA | 0 | 0 | 0 | 0 | 0 | 0 | 0 | 0 | 0 | 4 | 1 | 8 |
| TAG | 1 | 2 | 4 | 13 | 1 | 2 | 2 | 1 | 2 | 50 | 8 | 86 |
| Sum | 2 | 3 | 5 | 14 | 1 | 2 | 3 | 1 | 2 | 57 | 10 | 100 |

The results of Table 12 show that the PL fraction of the lipids present in the microbial biomass remained in the residual biomass and did not partition into the CO₂-extracted oil (i.e., 28 wt % PLs in the residual biomass versus 0.7 wt % in the extracted oil). Since the extracted oil was substantially free of PLs and enriched in TAGs relative to the microbial biomass, this oil was a refined lipid composition.

The refined lipid composition comprised 8 wt % FFAs, 5 wt % DAGs, and 86 wt % TAGs.

### Example 9

### Extraction of Residual Phospholipids with SCF CO₂/EtOH

The purpose of this Example was to demonstrate extraction of a first residual biomass sample with a mixture of SCF CO₂ and ethanol as the extractant to obtain a PL fraction and a second residual biomass sample.

An 18-mL extraction vessel fabricated from 316 SS tubing (1.27 cm o.d.×0.94 cm i.d.×26.0 cm long) was charged with 6.39 g of residual biomass from Example 3 (i.e., Yarrowia lipolytica strain Y8672 biomass following CO₂ extraction at 125 bar and 222 bar), which is referred to here as the “first residual biomass”. The material was flushed with CO₂, and then pressurized to 222 bar with a CO₂/ethanol mixture (the extractant) at 40° C. The CO₂ flow rate was 2.3 g/min and the ethanol flow rate was 0.12 g/min, giving an ethanol concentration of 5.0 wt % in the solvent fed to the extraction vessel. The first residual biomass was extracted at these conditions for 5.3 hrs, giving a final solvent-to-feed ratio of 120 g CO₂/ethanol per g residual biomass. The extraction yield of oil was 2.4 wt % from this previously-extracted material.

The Table below summarizes lipid analyses for the first residual biomass (the starting sample for this Example), the second residual biomass (the first residual biomass after extraction in this Example), and the extracted oil obtained by extraction of the first residual biomass.

TABLE 13
Example 9: Weight Percent Distribution of Lipid Components

| | | | | | 18:3 | | 20:3 | | 20:4 | | | |
|---|---|---|---|---|---|---|---|---|---|---|---|---|
| | 16:0 | 18:0 | 18:1 | 18:2 | (n-3) | 20:2 | (n-6) | 20:4 | (n-3) | 20:5 | | |
| Sample | palmitic | Stearic | Oleic | Linoleic | ALA | EDA | HGLA | ARA | ETA | EPA | other | Total |
| First Residual | 6 | 3 | 4 | 15 | 1 | 3 | 2 | 1 | 3 | 43 | 15 | 100 |
| Biomass | | | | | | | | | | | | |
| PL | 5 | 2 | 1 | 9 | 0 | 0 | 1 | 0 | 1 | 12 | 5 | 37 |
| DAG | 0 | 0 | 0 | 1 | 0 | 0 | 0 | 0 | 0 | 2 | 1 | 5 |
| FFA | 1 | 1 | 0 | 1 | 0 | 1 | 0 | 0 | 0 | 8 | 2 | 15 |
| TAG | 1 | 1 | 2 | 7 | 1 | 1 | 1 | 0 | 1 | 24 | 4 | 42 |
| Sum | 7 | 4 | 4 | 17 | 1 | 2 | 2 | 1 | 2 | 46 | 11 | 100 |
| Second | 6 | 3 | 4 | 16 | 1 | 2 | 2 | 1 | 4 | 48 | 8 | 100 |
| Residual | | | | | | | | | | | | |
| Biomass | | | | | | | | | | | | |
| PL | 5 | 2 | 1 | 8 | 0 | 0 | 1 | 0 | 1 | 10 | 5 | 35 |
| DAG | 0 | 0 | 0 | 1 | 0 | 0 | 0 | 0 | 0 | 2 | 1 | 5 |
| FFA | 1 | 1 | 0 | 1 | 0 | 1 | 0 | 0 | 0 | 7 | 2 | 14 |
| TAG | 1 | 1 | 2 | 7 | 1 | 1 | 1 | 0 | 1 | 26 | 4 | 45 |
| Sum | 7 | 4 | 4 | 17 | 1 | 2 | 2 | 1 | 2 | 45 | 12 | 100 |
| Extracted Oil | 8 | 5 | 4 | 15 | 1 | 3 | 3 | 1 | 2 | 43 | 14 | 100 |
| from First | | | | | | | | | | | | |
| Residual | | | | | | | | | | | | |
| Biomass (PL) | | | | | | | | | | | | |

The results of Table 13 show that the extracted oil was found to comprise essentially pure PLs. The extractions performed previously in Example 3 had already removed neutral lipids (i.e., TAGs, DAGs, and MAGs) and FFAs from the microbial biomass.

### Example 10

The purpose of this Example is to provide alternative microbial biomass comprising at least one PUFA that could be utilized in the extraction and fractionation methods described herein, to result in a refined lipid composition enriched in TAGs relative to the oil composition of the microbial biomass.

Although numerous oleaginous yeast genetically engineered for production of omega-3/omega-6 PUFAs are suitable microbial biomass according to the disclosure herein, representative strains of the oleaginous yeast Yarrowia lipolytica are described in Table 3. These include the following strains that have been deposited with the ATCC: Y. lipolytica strain Y2047 (producing ARA; ATCC Accession No. PTA-7186); Y. lipolytica strain Y2096 (producing EPA; ATCC Accession No. PTA-7184); Y. lipolytica strain Y2201 (producing EPA; ATCC Accession No. PTA-7185); Y. lipolytica strain Y3000 (producing DHA; ATCC Accession No. PTA-7187); Y. lipolytica strain Y4128 (producing EPA; ATCC Accession No. PTA-8614); Y. lipolytica strain Y4127 (producing EPA; ATCC Accession No. PTA-8802); Y. lipolytica strain Y8406 (producing EPA; ATCC Accession No. PTA-10025); Y. lipolytica strain Y8412 (producing EPA; ATCC Accession No. PTA-10026); and, Y. lipolytica strain Y8259 (producing EPA; ATCC Accession No. PTA-10027).

Thus, for example, Table 3 shows microbial hosts producing from 25.9% to 34% GLA of total fatty acids, from 10.9% to 14% ARA of total fatty acids, from 9% to 53.2% EPA of total fatty acids and 5.6% DHA of total fatty acids.

One of skill in the art will appreciate that the methodology of the present invention is not limited to microbial biomass demonstrating high-level EPA production but is equally suitable to microbial biomass demonstrating high-level production of alternate omega-3/omega-6 PUFAs or combinations or PUFAs thereof.

1. A method comprising the steps of:
a) processing an untreated disrupted microbial biomass having an oil composition comprising at least one polyunsaturated fatty acid with a solvent comprising liquid or supercritical fluid carbon dioxide to obtain:
(i) an extract comprising a lipid fraction substantially free of phospholipids; and,
(ii) a residual biomass comprising phospholipids; and,
b) fractionating the extract obtained in step (a), part (i) at least once to obtain a refined lipid composition comprising at least one polyunsaturated fatty acid, wherein the refined lipid composition is enriched in triacylglycerols relative to the oil composition of the untreated disrupted microbial biomass.

2. The method of claim 1, wherein the refined lipid composition enriched in triacylglycerols comprises at least one lipid component selected from the group consisting of:
a) diacylglycerols;
b) monoacylglycerols;
c) free fatty acids; and,
d) combinations thereof.

3. The method of claim 1, wherein the refined lipid composition enriched in triacylglycerols is enriched in at least one polyunsaturated fatty acid relative to the untreated disrupted microbial biomass.

4. The method of claim 1, further comprising a step selected from the group consisting of:
(1) fractionating the extract obtained in step (a), part (i) to obtain a refined lipid composition comprising at least one polyunsaturated fatty acid, wherein the refined lipid composition is enriched in lipid components selected from the group consisting of diacylglycerols, monoacylglycerols, free fatty acids and combinations thereof relative to the oil composition of the untreated disrupted microbial biomass; and,
(2) processing the residual biomass comprising phospholipids of step (a), part (ii) with an extractant to obtain a residual biomass extract consisting essentially of phospholipids.

5. The method of claim 1, wherein the processing of step (a) is done at a temperature from about 20° C. to about 100° C. and at a pressure from about 60 bar to about 800 bar.

6. The method of claim 1, wherein the fractionating of step (b) is performed by altering the temperature, the pressure, or the temperature and the pressure, of the fractionating conditions.

7. The method of claim 1, wherein:
a) the processing solvent of step (a) comprises supercritical fluid carbon dioxide; and,
b) the fractionating of step (b) is done at a temperature from about 35° C. to about 100° C. and at a pressure from about 80 bar to about 600 bar.

8. The method of claim 1, wherein the untreated disrupted microbial biomass comprises oleaginous microbial cells.

9. The method of either of claim 1 or 3, wherein the at least one polyunsaturated fatty acid is selected from the group consisting of linoleic acid, γ-linolenic acid, eicosadienoic acid, dihomo-γ-linolenic acid, arachidonic acid, docosatetraenoic acid, ω-6 docosapentaenoic acid, α-linolenic acid, stearidonic acid, eicosatrienoic acid, eicosatetraenoic acid, ω-3 docosapentaenoic acid, docosahexaenoic acid, eicosapentaenoic acid, and mixtures thereof.

10. The method of either of claims 1 or 4, wherein the residual biomass comprising phospholipids or the residual biomass extract consisting essentially of phospholipids is suitable for use as a component in an aquaculture feed.

11. The method of claim 1, wherein the untreated disrupted microbial biomass comprises at least 25 weight percent of eicosapentaenoic acid, measured as a weight percent of total fatty acids in the untreated disrupted microbial biomass.

12. A method comprising processing an untreated disrupted microbial biomass having an oil composition comprising at least one polyunsaturated fatty acid with a solvent comprising liquid or supercritical fluid carbon dioxide to obtain:
(i) an extract comprising a lipid fraction substantially free of phospholipids; and,
(ii) a residual biomass comprising phospholipids;
wherein said untreated disrupted microbial biomass is obtained from an oleaginous microorganism of the genus Yarrowia that accumulates in excess of 25% of its dry cell weight as oil; and,
wherein said oil composition comprising at least one polyunsaturated fatty acid comprises at least 25 weight percent of a polyunsaturated fatty acid having at least twenty carbon atoms and four or more carbon-carbon double bonds, measured as a weight percent of total fatty acids.

13. The method of claim 12, wherein the untreated disrupted microbial biomass is obtained from Yarrowia lipolytica and wherein the at least one polyunsaturated fatty acid comprises eicosapentaenoic acid.

14. The method of claim 12, wherein the residual biomass comprising phospholipids is processed with an extractant to obtain a residual biomass extract consisting essentially of phospholipids.

15. A phospholipid obtained from a recombinant microorganism engineered to produce at least 25 weight percent of eicosapentaenoic acid and no docosahexaenoic acid, each measured as a weight percent of total fatty acids, wherein said phospholipid comprises eicosapentaenoic acid and no docosahexaenoic acid.

16. The phospholipid of claim 15 wherein the recombinant microorganism is Yarrowia lipolytica.

17. A phospholipid obtained from a recombinant microorganism engineered to produce eicosapentaenoic acid, docosahexaenoic acid and omega-3 docosapentaenoic acid, wherein said phospholipid comprises eicosapentaenoic acid, docosahexaenoic acid and omega-3 docosapentaenoic acid.

18. The phospholipid of claim 17 wherein the recombinant microorganism is Yarrowia lipolytica.

19. Use of the phospholipid of any of claims 15-18 in formulating food, feed or a pharmaceutical composition.
